(19) United States
(12) Patent Application Publication
(10) Pub. No.: US 2023/0115322 A1
(43) Pub. Date: Apr. 13, 2023

(54) INCISION SIMULATION DEVICE, INCISION SIMULATION METHOD, AND PROGRAM

(71) Applicant: FUJIFILM Corporation, Tokyo (JP)

(72) Inventor: Futoshi SAKURAGI, Tokyo (JP)

(73) Assignee: FUJIFILM Corporation, Tokyo (JP)

(21) Appl. No.: 17/954,340

(22) Filed: Sep. 28, 2022

(30) Foreign Application Priority Data
Sep. 30, 2021 (JP) 2021-161791

(51) Int. Cl. G06T 7/00

(52) U.S. Cl. CPC G06T 7/0014

(57) ABSTRACT

An incision simulation device includes a processor configured to acquire a first incision line for a three-dimensional organ image that is a three-dimensional image showing an organ, acquire a first depth of incision to the first incision line, calculate a first excision region based on the first incision line and the first depth, acquire a second incision line for the three-dimensional organ image, acquire a second depth of incision to the second incision line, calculate a second excision region based on the first excision region, the second incision line, and the second depth, and identify a first region included in the first excision region and the second excision region, in the three-dimensional organ image.

### CROSS-REFERENCE TO RELATED APPLICATION

This application claims priority from Japanese Patent Application No. 2021-161791, filed Sep. 30, 2021, the disclosure of which is incorporated herein by reference in its entirety.

### BACKGROUND

### Technical Field

A technique of the present disclosure relates to an incision simulation device, an incision simulation method, and a non-transitory storage medium storing a program.

### Related Art

JP2014-018619A discloses a surgery support device comprising an image generation unit that generates an image showing an organ with an excision region specified in an aspect where a blood vessel region in the organ is visible, from a three-dimensional image of the organ, a depth input reception unit that receives an input for designating a depth of cutting, and a cut section setting unit that sets, as a cut section, a portion of a boundary surface between the excision region and a non-excision region as a region other than the excision region in the organ, within a range of the depth of cutting along the boundary surface from an outer edge of the boundary surface toward an inside, in which the image generation unit generates an image showing the organ in an aspect where only a partial blood vessel region present in a neighborhood region of the cut section in the blood vessel region of the organ is visible, from the three-dimensional image.

JP2007-222629A discloses a method for volume-rendering a digital medical image, the method including a step of providing a digital medical image volume, the image including a plurality of intensities on a three-dimensional grid of points, a step of providing a projection plane, the projection plane including a two-dimensional lattice of points, and rendering radiation is projected onto the projection plane from a viewpoint through the image volume, a step of advancing a sampling point along the radiation passing through the image volume, a step of creating an incision region in the image volume, a step of determining whether or not the sampling point is in the incision region, and a step of, in a case where the sampling point is in the incision region, using a first transfer function for a sample value interpolated from a first volume, in a case where the sampling point is outside the incision region, using a second transfer function to a sample value interpolated from a second volume, and accumulating an output of the transfer function.

JP2008-167793A discloses a method that performs surgery support through a medical image of a subject displayed on a display, in which images simulating a double-page spread state of a cut section by surgical instrument are created from three-dimensional image data of the subject and are displayed.

### SUMMARY

An aspect of the technique of the present disclosure provides an incision simulation device, an incision simulation method, and a non-transitory storage medium storing a program capable of identifying an inside of an excision region obtained by continuous incision.

A first aspect according to the technique of the present disclosure is an incision simulation device comprising a processor, in which the processor is configured to acquire a first incision line for a three-dimensional organ image that is a three-dimensional image showing an organ, acquire a first depth of incision to the first incision line, calculate a first excision region based on the first incision line and the first depth, acquire a second incision line for the three-dimensional organ image, acquire a second depth of incision to the second incision line, calculate a second excision region based on the first excision region, the second incision line, and the second depth, and identify a first region included in the first excision region and the second excision region, in the three-dimensional organ image.

A second aspect according to the technique of the present disclosure is an incision simulation method comprising acquiring a first incision line for a three-dimensional organ image that is a three-dimensional image showing an organ, acquiring a first depth of incision to the first incision line, calculating a first excision region based on the first incision line and the first depth, acquiring a second incision line for the three-dimensional organ image, acquiring a second depth of incision to the second incision line, calculating a second excision region based on the first excision region, the second incision line, and the second depth, and identifying a first region included in the first excision region and the second excision region, in the three-dimensional organ image.

A third aspect according to the technique of the present disclosure is a non-transitory storage medium storing a program that causes a computer to execute a process, the process comprising acquiring a first incision line for a three-dimensional organ image that is a three-dimensional image showing an organ, acquiring a first depth of incision to the first incision line, calculating a first excision region based on the first incision line and the first depth, acquiring a second incision line for the three-dimensional organ image, acquiring a second depth of incision to the second incision line, calculating a second excision region based on the first excision region, the second incision line, and the second depth, and identifying a first region included in the first excision region and the second excision region, in the three-dimensional organ image.

### BRIEF DESCRIPTION OF THE DRAWINGS

FIG. 1 is a conceptual diagram showing a schematic configuration of a medical service support device.

FIG. 2 is a block diagram showing an example of a hardware configuration of an electric system of the medical service support device.

FIG. 3 is a conceptual diagram showing an example of processing contents of an extraction unit.

FIG. 4 is a conceptual diagram showing an example of processing contents of a rendering unit.

FIG. 5 is a conceptual diagram showing an example of an aspect where rendering is performed on a three-dimensional organ image.

FIG. 6 is a conceptual diagram showing an example of an aspect where a first incision line is set.

FIG. 7 is a conceptual diagram showing an example of an aspect where a first depth is set.

FIG. 8 is a conceptual diagram showing an example of processing contents of an incision parameter acquisition unit, a first excision region calculation unit, and a region specification unit.

FIG. 9 is a conceptual diagram showing an example of an aspect where a second incision line is set.

FIG. 10 is a conceptual diagram showing an example of an aspect where a second depth is set.

FIG. 11 is a conceptual diagram showing an example of processing contents of the incision parameter acquisition unit, a second excision region calculation unit, and the region specification unit.

FIG. 12 is a screen diagram showing an example of an aspect where a rendering image in which a target region is brought into non-display is displayed on a display.

FIG. 13 is a screen diagram showing an example of an aspect where a rendering image in which a blood vessel system and the like are displayed is displayed on the display.

FIG. 14 is a flowchart illustrating an example of a flow of incision simulation processing.

FIG. 15 is a flowchart illustrating an example of the flow of the incision simulation processing.

FIG. 16 is a screen diagram showing an example of an aspect where a rendering image in which a blood vessel system and the like are displayed is displayed on the display.

FIG. 17 is a screen diagram showing an example of an aspect where a rendering image in which a blood vessel system and the like are displayed is displayed on the display.

FIG. 18 is a conceptual diagram showing a schematic configuration of a medical service support system.

### DETAILED DESCRIPTION

An example of an embodiment of an incision simulation device, an incision simulation method, and a program according to the technique of the present disclosure will be described with reference to the accompanying drawings.

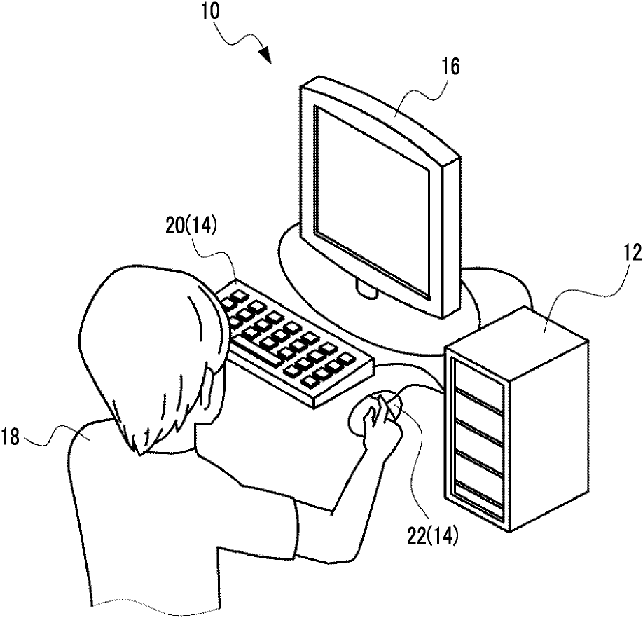

As shown in FIG. 1 as an example, a medical service support device 10 comprises an image processing device 12, a reception device 14, and a display 16, and is used by a user 18. Here, examples of the user 18 include a physician and a technician.

The reception device 14 is connected to the image processing device 12. The reception device 14 receives an instruction from the user 18. The reception device 14 has a keyboard 20, a mouse 22, and the like. In the example shown in FIG. 1, although the keyboard 20 and the mouse 22 are shown as the reception device 14, these are merely an example, any one of the keyboard 20 or the mouse 22 may be provided. Instead of the keyboard 20 and/or the mouse 22, at least one of an approach input device that receives an approach input, a voice input device that receives a voice input, or a gesture input device that receives a gesture input may be applied. At least one of the devices may be used as the reception device 14. The approach input device is, for example, a touch panel, a tablet, or the like. The reception device 14 and the image processing device 12 may be connected in a wired or wireless manner.

The display 16 is connected to the image processing device 12. Examples of the display 16 include an electro-luminescence (EL) display and a liquid crystal display. The display 16 displays various kinds of information (for example, an image and text) under the control of the image processing device 12.

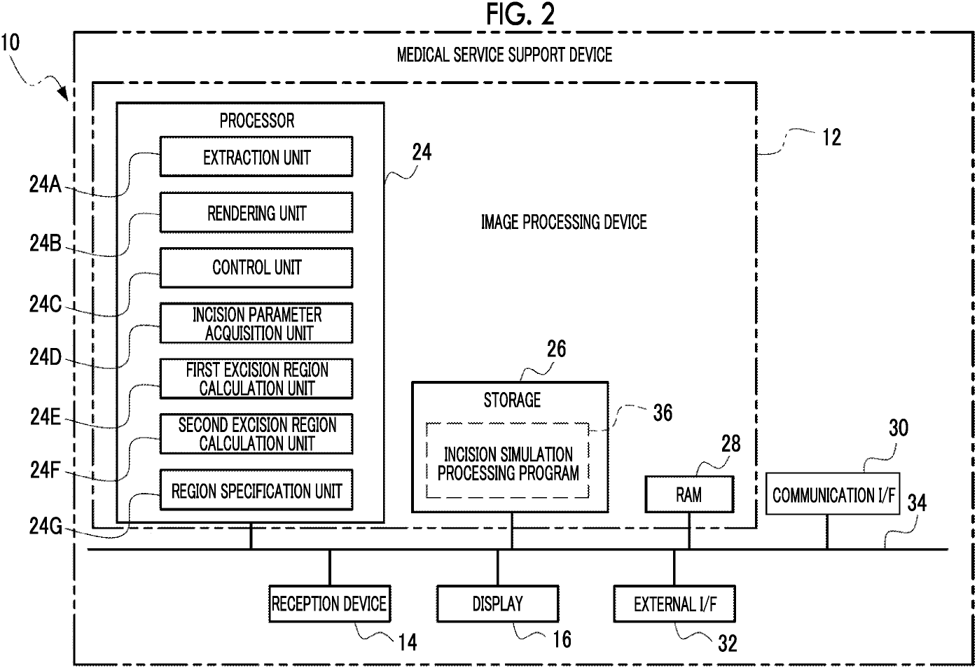

As shown in FIG. 2 as an example, the medical service support device 10 comprises a communication interface (I/F) 30, an external I/F 32, and a bus 34, in addition to the image processing device 12, the reception device 14, and the display 16.

The image processing device 12 is an example of an “incision simulation device” and a “computer” according to the technique of the present disclosure, and comprises a processor 24, a storage 26, and a random access memory (RAM) 28. The processor 24, the storage 26, the RAM 28, the communication I/F 30, and the external I/F 32 are connected to the bus 34.

A memory is connected to the processor 24. The memory includes the storage 26 and the RAM 28. The processor 24 has, for example, a central processing unit (CPU) and a graphics processing unit (GPU). The GPU operates under the control of the CPU and is responsible for execution of processing regarding an image. The processing regarding an image includes, for example, incision simulation processing described below.

The storage 26 is a nonvolatile storage device that stores various programs, various parameters, and the like. Examples of the storage 26 include a flash memory (for example, an electrically erasable and programmable read only memory (EEPROM) and/or a solid state drive (SSD)) and/or a hard disk drive (HDD).

The RAM 28 is a memory in which information is temporarily stored and is used as a work memory by the processor 24. Examples of the RAM 28 include a dynamic random access memory (DRAM) and a static random access memory (SRAM).

The communication I/F 30 is connected to a network (not shown). The network may be configured with at least one of a local area network (LAN) or a wide area network (WAN). An external device (not shown) and the like are connected to the network, and the communication I/F 30 controls transfer of information with an external communication device through the network. The external communication device may include, for example, at least one of a computed tomography (CT) apparatus, a magnetic resonance imaging (MRI) apparatus, a personal computer, or a smart device. For example, the communication I/F 30 transmits information depending on a request from the processor 24 to the external communication device through the network. The communication I/F 30 receives information transmitted from the external communication device and outputs the received information to the processor 24 through the bus 34.

The external I/F 32 controls transfer of various kinds of information with an external device (not shown) outside the medical service support device 10. The external device may be, for example, at least one of a smart device, a personal computer, a server, a universal serial bus (USB) memory, a memory card, or a printer. An example of the external I/F 32 is a USB interface. The external device is connected directly or indirectly to the USB interface.

Before surgery for removing a malignant tumor, such as lung cancer and/or liver cancer, from an organ, an excision region is determined and planned before surgery using a plurality of two-dimensional slice images or the like obtained by imaging a patient as a subject with a modality, such as a CT apparatus and/or an MRI apparatus, thereby increasing the safety of surgery.

Note that, actually, surgery may be performed while performing continuous incision, instead of excising from a planned incision line to a target excision region in one step. The continuous incision indicates a method in which primary incision is performed from a certain incision line, and then, incision is performed from a next incision line newly determined to a region after incision. In actual surgery, in many cases, a method in which continuous incision is performed, and finally, a target excision region is excised is employed. A simulation of such continuous incision is not taken into consideration hitherto, and there is room for improvement in an incision simulation.

Accordingly, in the present embodiment, to enable a simulation of continuous incision, as shown in FIG. 2 as an example, incision simulation processing is executed by the processor 24. An incision simulation processing program 36 is stored in the storage 26. The processor 24 reads out the incision simulation processing program 36 from the storage 26 and executes the read-out incision simulation processing program 36 on the RAM 28 to execute the incision simulation processing. The incision simulation processing is realized by the processor 24 functioning as an extraction unit 24A, a rendering unit 24B, a control unit 24C, an incision parameter acquisition unit 24D, a first excision region calculation unit 24E, a second excision region calculation unit 24F, and a region specification unit 24G. The incision simulation processing program 36 is an example of a “program” according to the technique of the present disclosure.

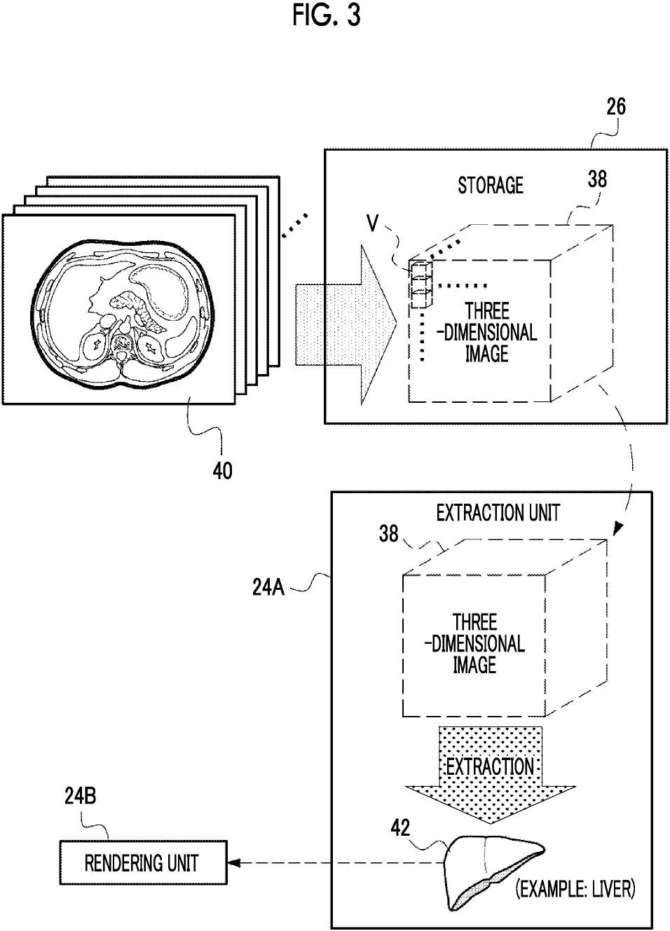

As shown in FIG. 3 as an example, a three-dimensional image 38 is stored in the storage 26. The three-dimensional image 38 is an image obtained by piling a plurality of two-dimensional slice images 40 obtained by imaging a patient with a modality and dividing the pile of images into voxels V. An example of the modality is a CT apparatus. The CT apparatus is merely an example, and other examples of the modality are an MRI apparatus, an ultrasound diagnostic apparatus, and the like. In the example shown in FIG. 3, although a two-dimensional slice image of a transverse plane is shown as a two-dimensional slice image 40, the technique of the present disclosure is not limited thereto, and a two-dimensional slice image of a coronal plane may be used or a two-dimensional slice image of a sagittal plane may be used. A position of each of all voxels V defining the three-dimensional image is specified by three-dimensional coordinates. Each voxel V is given, for example, a white and black shading value, such as a CT value.

The extraction unit 24A acquires the three-dimensional image 38 from the storage 26 and extracts a three-dimensional organ image 42 from the acquired three-dimensional image 38. The three-dimensional organ image 42 is a three-dimensional image showing an organ. For example, the three-dimensional image 38 includes a plurality of three-dimensional organ images 42, and each of the three-dimensional organ images 42 is given a unique identifier. The three-dimensional organ image 42 is extracted from the three-dimensional image 38 in response to an instruction received by the reception device 14. For example, the extraction unit 24A extracts the three-dimensional organ image 42 corresponding to an identifier received by the reception device 14 from the three-dimensional image 38. In the example shown in FIG. 3, an image showing a liver is shown as an example of the three-dimensional organ image 42. A unique identifier of each organ may be given to each voxel V of the three-dimensional image 38, and opacity and color information of red (R), green (G), and blue (B) may be set in the identifier of each organ. With this, each voxel V is given data (hereinafter, referred to as “voxel data”), such as opacity depending on the corresponding organ and color information of red (R), green (G), and blue (B), in addition to white and black shading value information.

Here, although an image showing a liver is illustrated as an example of the three-dimensional organ image 42, this is merely an example, and an image showing another organ, such as a heart and/or a lung, may be used. A method in which the three-dimensional organ image 42 is extracted using the unique identifier is merely an example, and a method in which the three-dimensional organ image 42 designated by the user 18 using any method through the reception device 14 is extracted by the extraction unit 24A may be used or a method in which the three-dimensional organ image 42 is extracted by the extraction unit 24A using image recognition processing by an artificial intelligence (AI) system and/or a pattern matching system may be used. The three-dimensional organ image 42 is not limited to an image showing a single organ. For example, an image in which, in addition to a liver, a plurality of organs, such as a blood vessel adjacent to the liver, a bile duct, and a gallbladder, are extracted may be used.

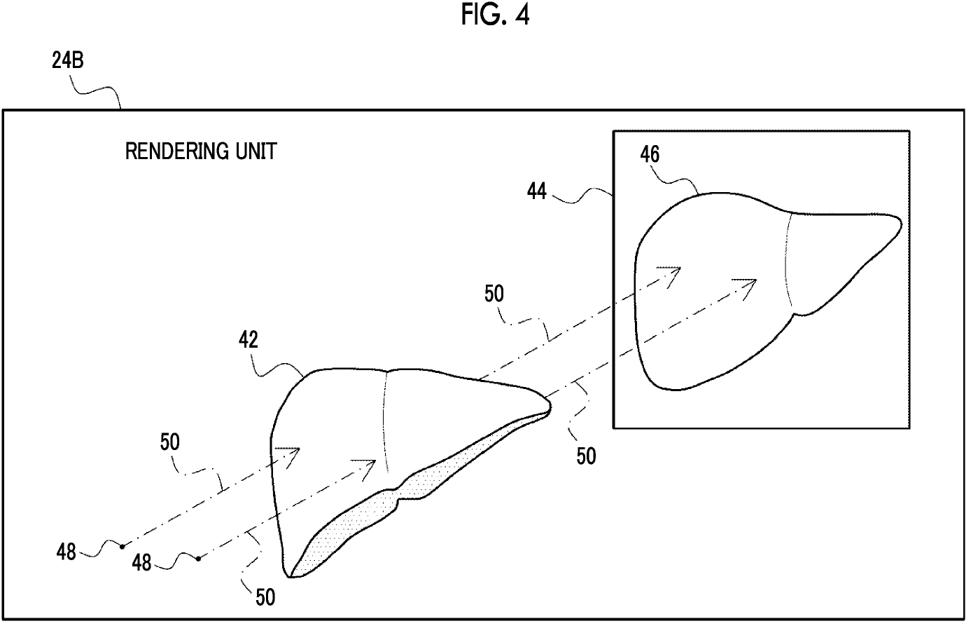

As shown in FIG. 4 as an example, the rendering unit 24B performs ray casting to render the three-dimensional organ image 42 on a projection plane 44 corresponding to a screen of the display 16. The rendering image 46 is projected onto the projection plane 44.

The projection plane 44 is, for example, a virtual plane that is defined with a resolution corresponding to a resolution of the screen of the display 16. Ray casting is performed by the rendering unit 24B, whereby a virtual ray 50 is projected from each viewpoint 48 corresponding to each pixel of the projection plane 44 onto the projection plane 44 through the three-dimensional organ image 42. A position of each viewpoint 48 with respect to the three-dimensional organ image 42 is changed in response to an instruction received by the reception device 14, and accordingly, a rendering image 46 in a case of observing the three-dimensional organ image 42 from various directions is projected onto the projection plane 44. The rendering image 46 projected onto the projection plane 44 is displayed on the display 16 or is stored in a predetermined storage device (for example, the storage 26), for example.

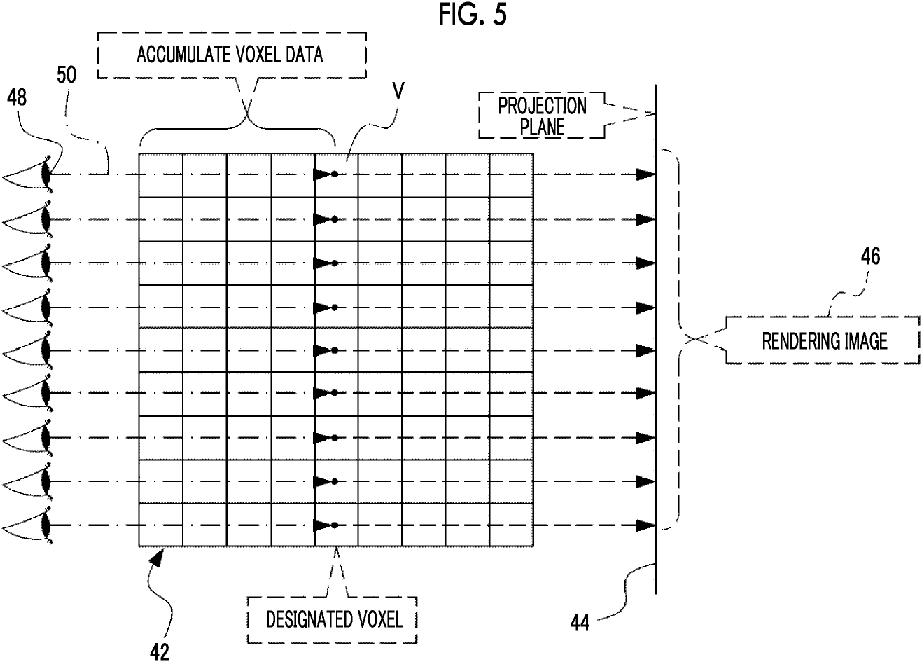

As shown in FIG. 5 as an example, the ray 50 projects data (hereinafter, referred to as “accumulated data”) obtained by accumulating voxel data obtained at sampling points (for example, points defined at intervals of one voxel) to a designated voxel, that is, to a voxel V at a designated position, onto the projection plane 44 while passing through the three-dimensional organ image 42. With this, each pixel of the projection plane 44 is given accumulated data as a pixel value. The rendering unit 24B generates the rendering image 46 depending on the accumulated data given to each pixel.

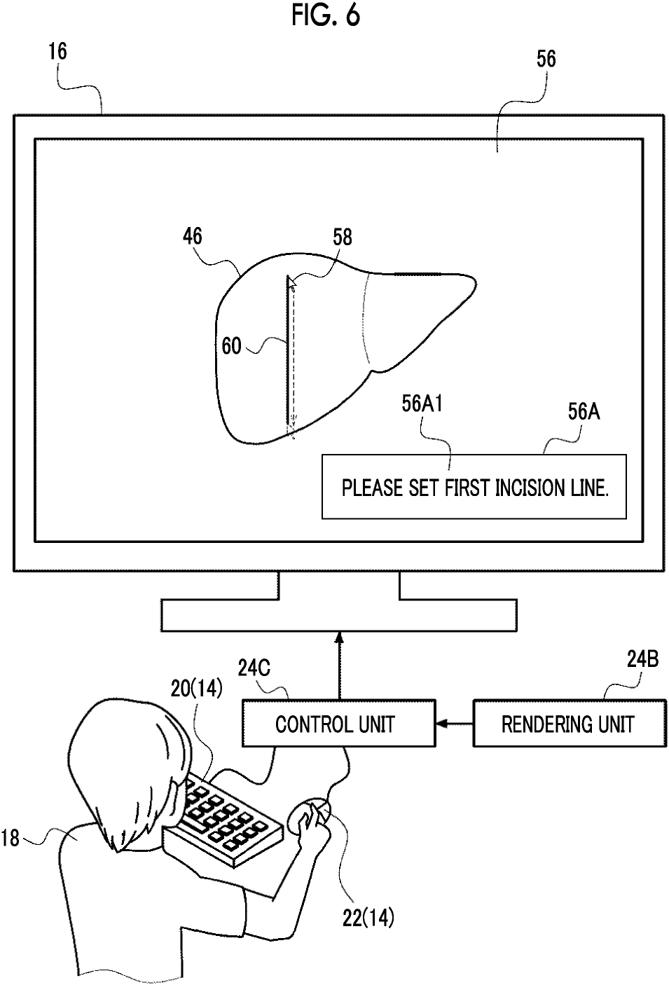

As shown in FIG. 6 as an example, the control unit 24C performs display control in response to an instruction received by the reception device 14 to display a screen 56 on the display 16. The control unit 24C performs various settings in response to an instruction received by the reception device 14.

On the screen 56, the rendering image 46 generated by the rendering unit 24B is displayed. A guide message display region 56A is included in the screen 56. A guide message 56A1 is displayed in the guide message display region 56A. The guide message 56A1 is a message for guiding setting of a first incision line 60 for the three-dimensional organ image 42 through the rendering image 46 to the user 18. In the example shown in FIG. 6, as an example of the guide message 56A1, a message “Please set first incision line.” is shown.

On the screen 56, a pointer 58 is displayed. The user 18 operates the pointer 58 through the reception device 14 (here, as an example, the mouse 22) to form the first incision line 60 for the rendering image 46. In the example shown in FIG. 6, as an example of the first incision line 60 formed for the rendering image 46 by the operation of the pointer 58, a linear line is shown. The first incision line 60 formed for the rendering image 46 is confirmed in response to an instruction received by the reception device 14.

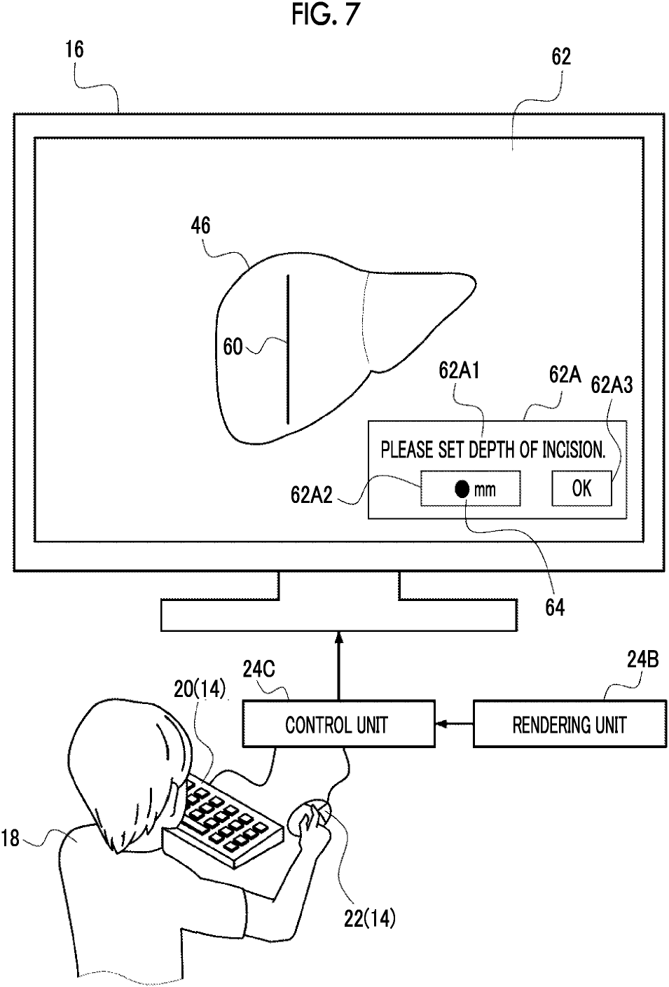

In a case where the setting of the first incision line 60 ends, as shown in FIG. 7 as an example, the screen displayed on the display 16 is switched from the screen 56 to a screen 62 by the control unit 24C. On the screen 62, the rendering image 46 where the first incision line 60 is drawn is displayed. A depth setting box 62A is included in the screen 62. The depth setting box 62A has a guide message 62A1, an input box 62A2, and an OK key 62A3.

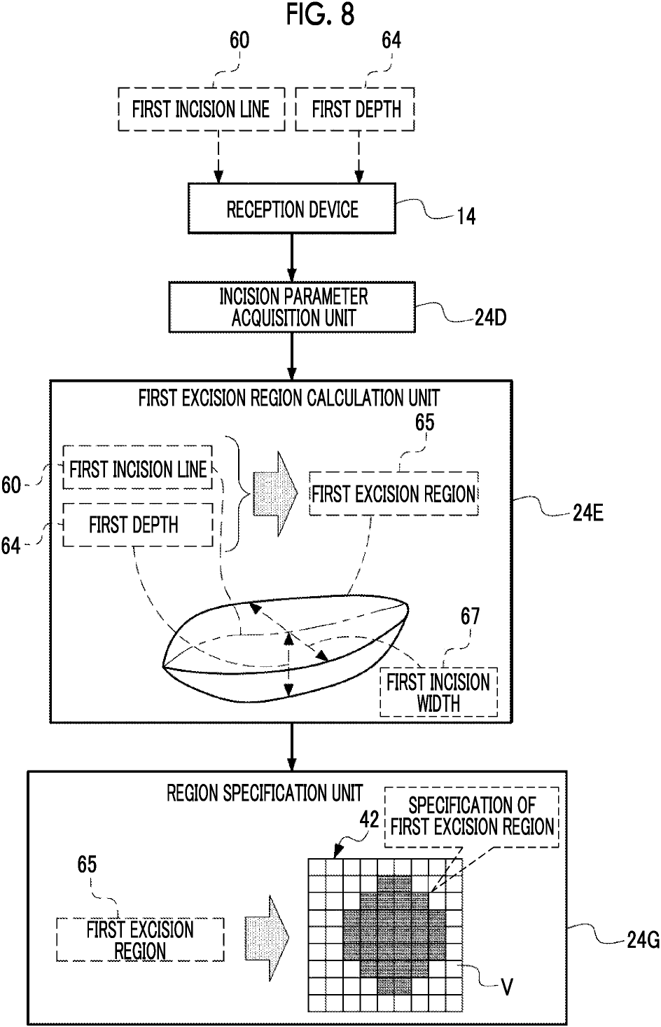

The guide message 62A1 is a message for guiding setting of a first depth 64 (see FIG. 8) of incision to the three-dimensional organ image 42, to the user 18. Hereinafter, for convenience of description, the first depth 64 of incision to the three-dimensional organ image 42 is simply referred to as a “first depth 64”. In the example shown in FIG. 7, a message “Please set depth of incision.” is shown. Here, the first depth 64 is a depth at a designated position on the first incision line 60, and is, for example, a depth in an intermediate portion of the first incision line 60. The intermediate portion of the first incision line 60 is, for example, a region including a middle point of the first incision line 60 in a case where a length of the first incision line 60 is equally divided into three portions. In particular, the first depth 64 is a depth at the middle point in the first incision line 60.

The input box 62A2 is a box to which the first depth 64 is input. For example, the first depth 64 is input to the input box 62A2 as a numerical value in units of millimeters. The user 18 inputs the first depth 64 to the input box 62A2 through the reception device 14 (here, as an example, the keyboard 20).

The OK key 62A3 is a soft key that is turned on in a case of confirming the first depth 64 input to the input box 62A2. The user 18 turns on the OK key 62A3 through the reception device 14 (here, as an example, the mouse 22) in a case where the first depth 64 is input to the input box 62A2. With this, the first depth 64 input to the input box 62A2 is confirmed.

In a case where the setting of the first depth 64 ends, as shown in FIG. 8 as an example, the incision parameter acquisition unit 24D acquires the first incision line 60 and the first depth 64 received by the reception device 14. Hereinafter, for convenience of description, in a case where there is no need for distinction between the first incision line 60 and the first depth 64, the first incision line 60 and the first depth 64 are referred to as “incision parameters” without any reference sign.

As shown in FIG. 8 as an example, the first excision region calculation unit 24E acquires the first incision line 60 and the first depth 64 from the incision parameter acquisition unit 24D. The first excision region calculation unit 24E calculates a first excision region 65 based on the first incision line 60 and the first depth 64.

Specifically, the first excision region calculation unit 24E calculates a depth at each position of the first incision line 60 based on the first depth 64. In this case, the depth at each position of the first incision line 60 is shallower from the first depth 64 toward an end of the first incision line 60 at each position on the first incision line 60. In the example shown in FIG. 8, change in depth at each position of the first incision line 60 is nonlinear change. That is, the depth is changed in a curved shape as viewed from a normal direction of a plane defined by the first incision line 60 and the first depth 64. The nonlinear change is merely an example. For example, the change in depth at each position of the first incision line 60 may be linear change. That is, the depth may be changed in a linear shape as viewed from the normal direction of the plane defined by the first incision line 60 and the first depth 64. The depth at each position of the first incision line 60 is calculated, for example, from a first incision line depth arithmetic expression (not shown) by the first excision region calculation unit 24E. The first incision line depth arithmetic expression is an arithmetic expression that has a parameter (for example, a length of the first incision line 60) for specifying a geometrical characteristic of the first incision line 60 and the first depth 64 as independent variables, and has the depth at each position of the first incision line 60 as a dependent variable.

The first excision region calculation unit 24E calculates the first excision region 65 based on the first incision line 60, the first depth 64, and the depth at each position of the first incision line 60. The first excision region 65 is calculated, for example, from a first excision region arithmetic expression (not shown) by the first excision region calculation unit 24E. The first excision region arithmetic expression is an arithmetic expression that has the parameter (for example, the length of the first incision line 60) for specifying the geometrical characteristic of the first incision line 60, the first depth 64, and the depth at each position of the first incision line 60 as independent variables, and has the first excision region 65 as a dependent variable. The calculation of the first excision region 65 indicates, for example, calculation of a parameter (for example, three-dimensional coordinates specifying an outer edge of the first excision region 65 in the three-dimensional organ image 42) for specifying a geometrical characteristic of the first excision region 65.

The first excision region calculation unit 24E calculates a first incision width 67 that is an incision width at each position of the first incision line 60, based on the depth at each position of the first incision line 60. The first incision width 67 is narrower from a position of the first depth 64 toward the end of the first incision line 60 at each position on the first incision line 60. In the example shown in FIG. 8, the first incision width 67 at each position of the first incision line 60 is changed in a nonlinear shape. That is, in a case where the first excision region 65 is viewed in plan view, the width is changed in a curved shape. The nonlinear change is merely an example. For example, the first incision width 67 may be changed in a linear shape. That is, in a case where the first excision region 65 is viewed in plan view, the width may be changed in a linear shape. The first incision width 67 is calculated, for example, from a first incision width arithmetic expression (not shown) by the first excision region calculation unit 24E. The first incision width arithmetic expression is an arithmetic expression that has the parameter (for example, the length of the first incision line 60) for specifying the geometrical characteristic of the first incision line 60 and the depth at each position of the first incision line 60 as independent variables, and has the first incision width 67 as a dependent variable.

The first excision region calculation unit 24E calculates the first excision region 65 based on the first incision line 60, the first depth 64, and the first incision width 67. The first excision region calculation unit 24E adds the first incision width 67 as an independent variable and calculates the first excision region 65 as a dependent variable in the above-described first excision region arithmetic expression.

The first excision region calculation unit 24E calculates a depth at each position of the first incision width 67 based on the depth at each position of the first incision line 60. The depth at each position of the first incision width 67 is shallower from a position on the first incision line 60 toward an end of the first incision width 67. In the example shown in FIG. 8, change in depth at each position of the first incision width 67 is nonlinear change. That is, the depth is changed in a curved shape as viewed from a normal direction of a plane perpendicular to the first incision line 60. The nonlinear change is merely an example. For example, the change in depth at each position of the first incision width 67 may be linear. That is, the depth may be changed in a curved shape as viewed from the normal direction of the plane perpendicular to the first incision line 60. The depth at each position of the first incision width 67 is calculated, for example, from a first incision width depth arithmetic expression (not shown). The first incision width depth arithmetic expression is an arithmetic expression that has the first incision width 67, and a depth at an intersection of the first incision width 67 for which the depth is be calculated and the first incision line 60 as independent variables, and has the depth at each position of the first incision width 67 as a dependent variable.

The first excision region calculation unit 24E calculates the first excision region 65 based on the first incision line 60, the first depth 64, and the depth at each position of the first incision width 67. The first excision region calculation unit 24E adds the depth at each position of the first incision width 67 as an independent variable and calculates the first excision region 65 as a dependent variable in the above-described first excision region arithmetic expression.

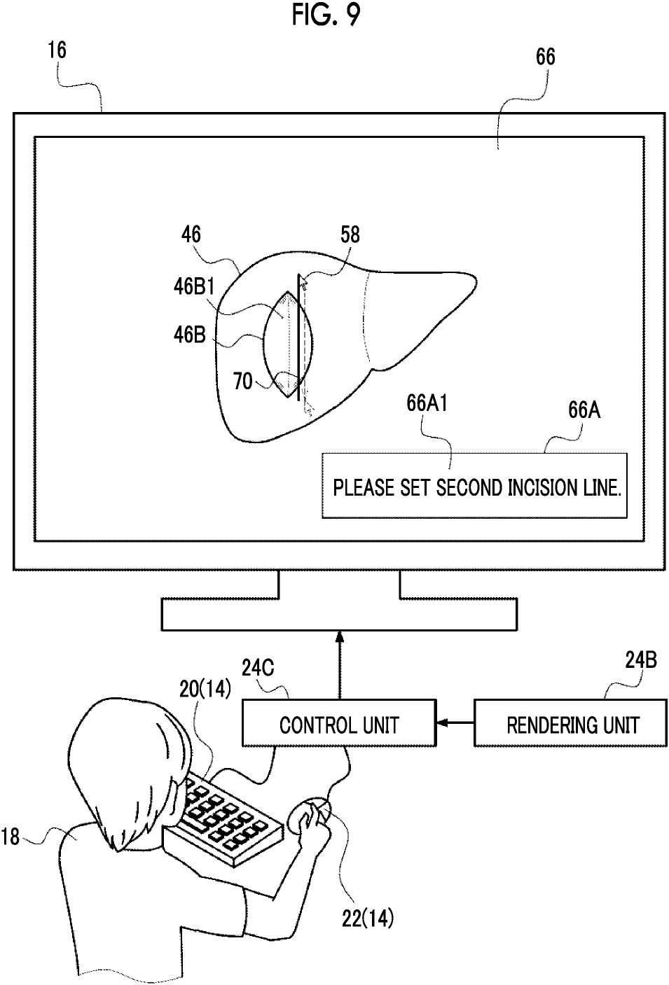

The region specification unit 24G specifies the first excision region 65 from the three-dimensional organ image 42 using a calculation result by the first excision region calculation unit 24E. The specification of the first excision region 65 means, for example, decision of three-dimensional coordinates specifying the position of the first excision region 65 in the three-dimensional organ image 42. The three-dimensional coordinates specifying the position of the first excision region 65 in the three-dimensional organ image 42 are decided, for example, by being calculated following an arithmetic expression that has the first incision line 60, the first depth 64, the first incision width 67, the depth at each position of the first incision line 60, the depth at each position of the first incision width 67, the first excision region 65, and the like as independent variables, and has three-dimensional coordinates specifying the position of the outer edge of the first excision region 65 in the three-dimensional organ image 42 as a dependent variable. In a case where the first excision region 65 is specified by the region specification unit 24G, as shown in FIG. 9 as an example, the rendering unit 24B performs rendering to the inside of the first excision region 65 (for example, an incision plane (that is, an exposed region) of the first excision region 65). With this, a rendering image 46B1 that is a rendering image showing an incision plane of the first excision region 65 is generated in a region 46B in the rendering image 46 corresponding to the outer edge of the first excision region 65.

As shown in FIG. 9 as an example, the control unit 24C performs display control such that the rendering image 46 including the rendering image 46B 1 is displayed on the display 16. The control unit 24C switches the displayed screen from the screen 62 (see FIG. 7) to a screen 66. On the screen 66, the rendering image 46B 1 generated by the rendering unit 24B is displayed. A guide message display region 66A is included in the screen 66. A guide message 66A1 is displayed in the guide message display region 66A. The guide message 66A1 is a message for guiding setting of the second incision line 70 for the three-dimensional organ image 42 through the rendering image 46 to the user 18. In the example shown in FIG. 9, as an example of the guide message 66A1, a message “Please set second incision line.” is shown.

The pointer 58 is displayed on the screen 66. The user 18 operates the pointer 58 through the reception device 14 (here, as an example, the mouse 22) to form the second incision line 70 for the rendering image 46. In the example shown in FIG. 9, as an example of the second incision line 70 formed for the rendering image 46 by the operation of the pointer 58, a linear line is shown. The second incision line 70 formed for the rendering image 46 is confirmed in response to an instruction received by the reception device 14.

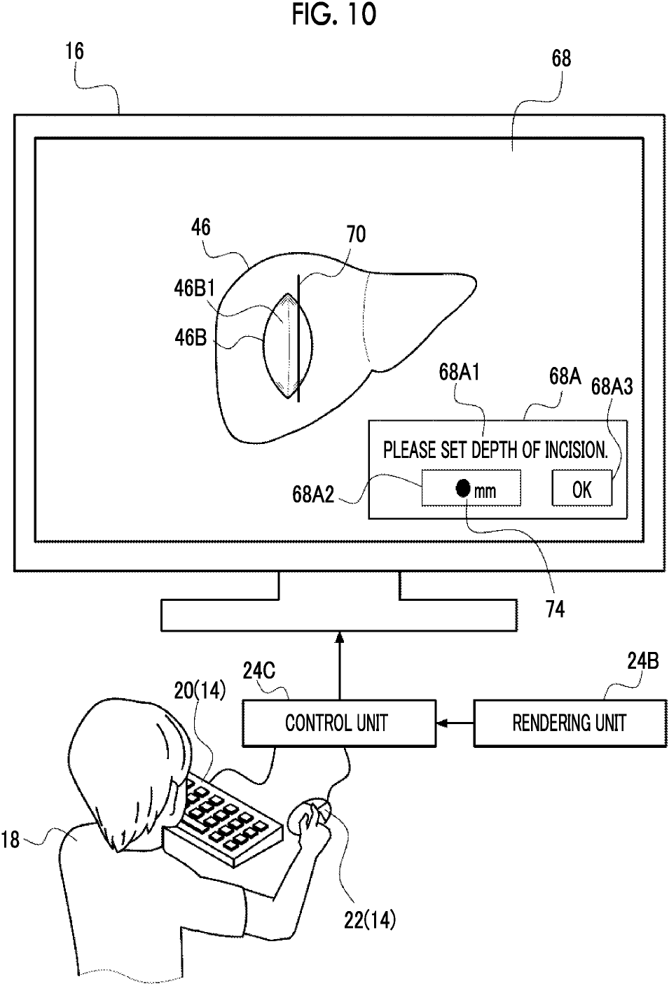

In a case where the setting of the second incision line 70 ends, as shown in FIG. 10 as an example, the screen displayed on the display 16 is switched from the screen 66 to a screen 68 by the control unit 24C. On the screen 68, the rendering image 46 where the second incision line 70 is drawn is displayed. A depth setting box 68A is included in the screen 68. The depth setting box 68A has a guide message 68A1, an input box 68A2, and an OK key 68A3.

The guide message 68A1 is a message for guiding setting of a second depth 74 (hereinafter, simply referred to as a “second depth 74”) of incision to the three-dimensional organ image 42 to the user 18. In the example shown in FIG. 10, a message “Please set depth of incision.” is shown. Here, the second depth 74 is a depth at a designated position on the second incision line 70 with a surface after the excision of the first excision region 65 as a starting point. The second depth 74 is, for example, a depth in an intermediate portion of the second incision line 70. The intermediate portion of the second incision line 70 is, for example, a region including a middle point of the second incision line 70 in a case where a length of the second incision line 70 is equally divided into three portions. In particular, the second depth 74 is a depth at the middle point in the second incision line 70.

The input box 68A2 is a box to which the second depth 74 is input. For example, the second depth 74 is input to the input box 68A2 as a numerical value in units of millimeters. The user 18 inputs the second depth 74 to the input box 68A2 through the reception device 14 (here, as an example, the keyboard 20).

The OK key 68A3 is a soft key that is turned on in a case of confirming the second depth 74 input to the input box 68A2. The user 18 turns on the OK key 68A3 through the reception device 14 (here, as an example, the mouse 22) in a case where the second depth 74 is input to the input box 68A2. With this, the second depth 74 input to the input box 68A2 is confirmed.

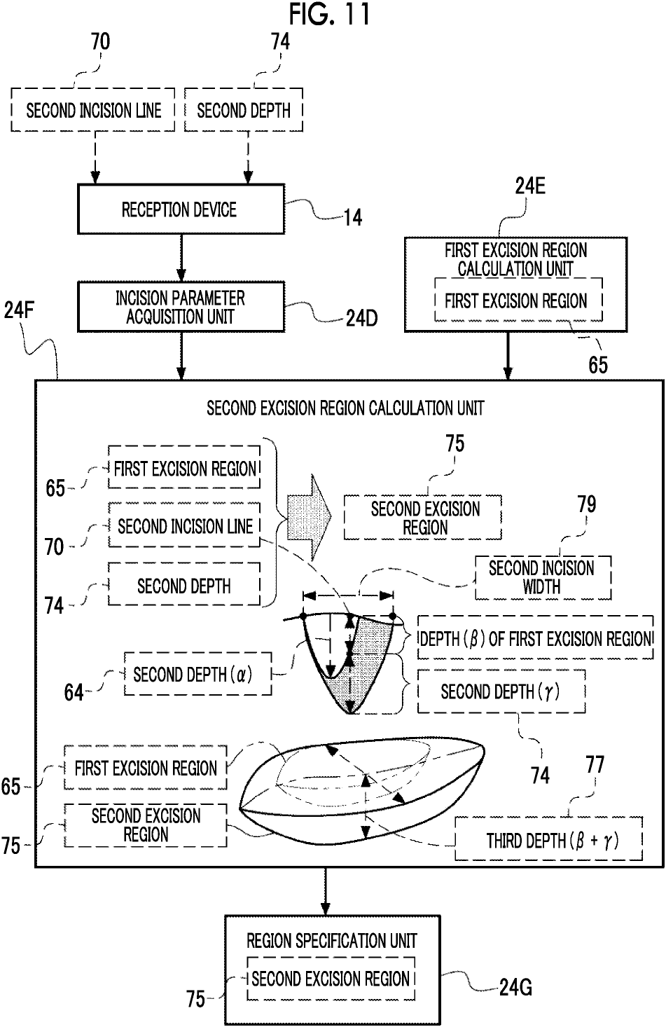

In a case where the setting of the second depth 74 ends, as shown in FIG. 11 as an example, the incision parameter acquisition unit 24D acquires the second incision line 70 and the second depth 74 received by the reception device 14. Hereinafter, for convenience of description, in a case where there is no need for distinction between the second incision line 70 and the second depth 74, the second incision line 70 and the second depth 74 are referred to as “incision parameters” without any reference sign.

As shown in FIG. 11 as an example, the second excision region calculation unit 24F acquires the second incision line 70 and the second depth 74 from the incision parameter acquisition unit 24D. The second excision region calculation unit 24F acquires the first excision region 65 calculated in the first excision region calculation unit 24E. The second excision region calculation unit 24F calculates a second excision region 75 based on the first excision region 65, the second incision line 70, and the second depth 74.

Incidentally, in a case where a continuous incision simulation is performed, the second excision region 75 is specified for the three-dimensional organ image 42 after the first excision region 65 is specified. Note that a space (that is, a hollow) corresponding to the first excision region 65 occurs in the three-dimensional organ image 42 where the first excision region 65 is excised. For this reason, in the continuous incision simulation, in a case where calculation for specifying the second excision region 75 including change in surface shape, such as a hollow, is performed, a calculation cost may be increased.

Accordingly, in the present embodiment, as an example, processing shown in FIG. 11 is executed by the processor 24. The second excision region calculation unit 24F calculates a third depth 77 based on a depth of the first excision region 65 and the second depth 74. The depth of the first excision region 65 is a distance β from a surface of the organ shown in the three-dimensional organ image 42 before the first excision region 65 is excised to a surface of the organ shown in the three-dimensional organ image 42 after the first excision region 65 is excised. As described above, the second depth 74 is a depth γ at the designated position on the second incision line 70 with a surface after the excision of the first excision region 65 as a starting point. The third depth 77 is, for example, a sum (that is, β + γ) of the depth of the first excision region 65 and the second depth 74. The second excision region calculation unit 24F calculates the second excision region 75 based on the second incision line 70 and the third depth 77.

Specifically, the second excision region calculation unit 24F calculates a depth at each position of the second incision line 70 based on the third depth 77. In this case, the depth at each position of the second incision line 70 is shallower from the third depth 77 at each position on the second incision line 70 to an end of the second incision line 70. In the example shown in FIG. 11, change in depth at each position of the second incision line 70 is nonlinear change. That is, the depth is changed in a curved shape as viewed from a normal direction of a plane defined by the second incision line 70 and the third depth 77. The nonlinear change is merely an example. For example, the change in depth at each position of the second incision line 70 may be linear. That is, the depth may be changed in a linear shape as viewed from the normal direction of the plane defined by the second incision line 70 and the third depth 77. The depth at each position of the second incision line 70 is calculated, for example, from a second incision line depth arithmetic expression (not shown) by the second excision region calculation unit 24F. The second incision line depth arithmetic expression is an arithmetic expression that has a parameter (for example, a length of the second incision line 70) for specifying a geometrical characteristic of the second incision line 70 and the third depth 77 as independent variables, and has the depth at each position of the second incision line 70 as a dependent variable. The second incision line depth arithmetic expression may be the same arithmetic expression as the first incision line depth arithmetic expression. Specifically, the parameter for specifying the geometrical characteristic of the second incision line 70 may be input to the independent variable of the parameter for specifying the geometrical characteristic of the first incision line 60, the third depth 77 may be input to the independent variable of the first depth 64, and the depth at each position of the second incision line 70, instead of the depth at each position of the first incision line 60, may be output as the dependent variable.

The second excision region calculation unit 24F calculates the second excision region 75 based on second incision line 70, the third depth 77, and the depth at each position of the second incision line 70. The second excision region 75 is calculated, for example, from a second excision region arithmetic expression (not shown) by the second excision region calculation unit 24F. The second excision region arithmetic expression is an arithmetic expression that has the parameter (for example, the length of the second incision line 70) for specifying the geometrical characteristic of the second incision line 70, the third depth 77, and the depth at each position of the second incision line 70 as independent variables, and has the second excision region 75 as a dependent variable. Here, the calculation of the second excision region 75 indicates, for example, calculation of the parameter (for example, three-dimensional coordinates specifying an outer edge of the second excision region 75 in the three-dimensional organ image 42) for specifying the geometrical characteristic of the second excision region 75. The second excision region arithmetic expression may be the same arithmetic expression as the first excision region arithmetic expression. Specifically, the parameter for specifying the geometrical characteristic of the second incision line 70 may be input to the independent variable of the parameter for specifying the geometrical characteristic of the first incision line 60, the third depth 77 may be input to the independent variable of the first depth 64, the depth at each position of the second incision line 70 may be input to the independent variable of the depth at each position of the first incision line 60, and the second excision region 75, instead of the first excision region 65, may be output as the dependent variable.

The second excision region calculation unit 24F calculates a second incision width 79 that is an incision width at each position of the second incision line 70, based on the depth at each position of the second incision line 70. The second incision width 79 is narrower from a position of the third depth 77 toward the end of the second incision line 70 at each position on the second incision line 70. In the example shown in FIG. 11, the second incision width 79 at each position of the second incision line 70 is changed in a nonlinear shape. That is, the width is changed in a curved shape in a case where the second excision region 75 is viewed in plan view. The nonlinear change is merely an example. For example, the second incision width 79 may be changed in a linear shape. That is, the width may be changed in a linear shape in a case where the second excision region 75 is viewed in plan view. The second incision width 79 is calculated, for example, from a second incision width arithmetic expression (not shown) by the second excision region calculation unit 24F. The second incision width arithmetic expression is an arithmetic expression that has the parameter (for example, the length of the second incision line 70) for specifying the geometrical characteristic of the second incision line 70 and the depth at each position of the second incision line 70 as independent variables, and has the second incision width 79 as a dependent variable. The second incision width arithmetic expression may be the same arithmetic expression as the first incision width arithmetic expression. Specifically, the parameter for specifying the geometrical characteristic of the second incision line 70 may be input to the independent variable of the parameter for specifying the geometrical characteristic of the first incision line 60, the depth at each position of the second incision line 70 may be input to the independent variable of the depth at each position of the first incision line 60, and the second incision width 79, instead of the first incision width 67, may be output as the dependent variable.

The second excision region calculation unit 24F calculates the second excision region 75 based on the parameter (for example, the length of the second incision line 70) for specifying the geometrical characteristic of the second incision line 70, the third depth 77, and the second incision width 79. The second excision region calculation unit 24F adds the second incision width 79 as an independent variable and calculates the second excision region 75 as a dependent variable in the above-described second excision region arithmetic expression. The second excision region arithmetic expression may be the same arithmetic expression as the first excision region arithmetic expression. Specifically, the second incision width 79 may be input to the independent variable of the first incision width 67, and the second excision region 75, instead of the first excision region 65, may be output as the dependent variable.

The second excision region calculation unit 24F calculates a depth at each position of the second incision width 79 based on the depth at each position of the second incision line 70. The depth at each position of the second incision width 79 is shallower from a position on the second incision line 70 toward an end of the second incision width 79. In the example shown in FIG. 11, change in depth at each position of the second incision width 79 is nonlinear change. That is, the depth is changed in a curved shape as viewed from a normal direction of a plane perpendicular to the second incision line 70. The nonlinear change is merely an example. For example, the change in depth at each position of the second incision width 79 may be linear. That is, the depth may be changed in a linear shape as viewed from the normal direction of the plane perpendicular to the second incision line 70. The depth at each position of the second incision width 79 is calculated, for example, from a second incision width depth arithmetic expression (not shown). The second incision width depth arithmetic expression is an arithmetic expression that has the second incision width 79 and a depth of the second incision line 70 at an intersection of the second incision width 79 for which the depth is to be calculated and the second incision line 70 as independent variables, and has the depth at each position of the second incision width 79 as a dependent variable. The second incision width depth arithmetic expression is the same arithmetic expression as the first incision width depth arithmetic expression. Specifically, the second incision width 79 may be input to the independent variable of the first incision width 67, the depth of the second incision line 70 at the intersection of the second incision width 79 for which the depth is to be calculated and the second incision line 70 may be input to the independent variable of the depth at the intersection of the first incision width 67 for which the depth is to be calculated and the first incision line 60, and the depth at each position of the second incision width 79, instead of the depth at each position of the first incision width 67, may be output as the dependent variable.

The second excision region calculation unit 24F calculates the second excision region 75 based on the parameter (for example, the length of the second incision line 70) for specifying the geometrical characteristic of the second incision line 70, the third depth 77, and the depth at each position of the second incision width 79. The second excision region calculation unit 24F adds the depth at each position of the second incision width 79 as an independent variable and calculates the parameter (for example the three-dimensional coordinates specifying the outer edge of the second excision region 75 in the three-dimensional organ image 42) for specifying the geometrical characteristic of the second excision region 75 as a dependent variable in the above-described second excision region arithmetic expression. The second excision region arithmetic expression may be the same arithmetic expression as the first excision region arithmetic expression. Specifically, the depth at each position of the second incision width 79 may be input to the independent variable of the depth at each position of the first incision width 67, and the second excision region 75, instead of the first excision region 65, may be output as the dependent variable.

The region specification unit 24G specifies the second excision region 75 from the three-dimensional organ image 42 using a calculation result by the second excision region calculation unit 24F. The specification of the second excision region 75 means, for example, the decision of the three-dimensional coordinates specifying the position of the second excision region 75 in the three-dimensional organ image 42. The three-dimensional coordinates specifying the position of the second excision region 75 in the three-dimensional organ image 42 is decided, for example, by being calculated following an arithmetic expression that has the second incision line 70, the third depth 77, the second incision width 79, the depth at each position of the second incision line 70, the depth at each position of the second incision width 79, the second excision region 75, and the like as independent variables, and has the three-dimensional coordinates specifying the position of the outer edge of the second excision region 75 in the three-dimensional organ image 42 as a dependent variable.

In a case where the second excision region 75 is specified by the region specification unit 24G, the region specification unit 24G specifies a target region 80 included in the first excision region 65 and the second excision region 75. The specification of the target region 80 indicates specification of three-dimensional coordinates of a plurality of voxels composing the target region 80. The target region 80 is a region that composes a part or the whole of a region including the first excision region 65 the second excision region 75. The target region 80 is an example of a “first region” according to the technique of the present disclosure.

In the example shown in FIG. 11, an example where the entire first excision region 65 is included in the second excision region 75, and the target region 80 is the same region as the second excision region 75 is shown. The target region 80 may be specified based on a reception result of a range in the second excision region 75 designated by the user 18, through the reception device 14 or may be specified following a condition (for example, a range determined in advance depending on a type of organ) determined in advance.

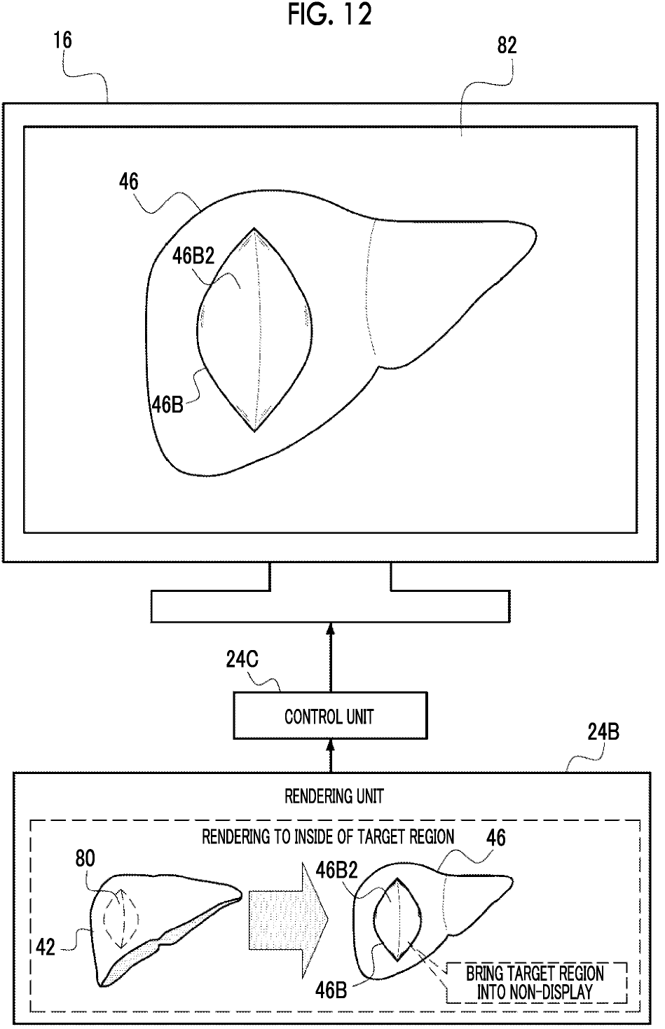

The rendering unit 24B generates a rendering image 46 in a state in which the target region 80 is brought into non-display. As shown in FIG. 12 as an example, the rendering unit 24B acquires the target region 80 specified by the region specification unit 24G. The rendering unit 24B performs rendering to the inside of the target region 80 (for example, in a case where the target region 80 is the same as the second excision region 75, an incision plane (that is, an exposed region) of the second excision region 75). With this, a rendering image 46B2 that shows a surface in contact with an outer edge of the target region 80 is generated in a region 46B in the rendering image 46 corresponding to the outer edge of the target region 80. For example, in a case where the target region 80 is the same as the second excision region 75, the rendering image 46B2 is a rendering image that shows the incision plane of the second excision region 75.

The control unit 24C performs display control such that the rendering image 46 including the rendering image 46B2 is displayed on the display 16. As shown in FIG. 12 as an example, on a screen 82, the rendering image 46 including the rendering image 46B2 is displayed.

The region specification unit 24G specifies a region that shows each of a blood vessel system, a lymphatic system, a nervous system, and/or a lesion part (for example, a tumor) inside the target region 80 in the three-dimensional organ image 42. The specification of the blood vessel system, the lymphatic system, the nervous system, and/or the lesion part indicates specification of three-dimensional coordinates of a plurality of voxels composing the respective regions specified as the blood vessel system, the lymphatic system, the nervous system, and/or the lesion part. The region specification unit 24G executes image recognition processing on the inside of the target region 80 in the three-dimensional organ image 42 to specify the blood vessel system, the lymphatic system, the nervous system, and/or the lesion part. The image recognition processing is not particularly limited, and for example, a method in which the regions showing the blood vessel system, the lymphatic system, the nervous system, and/or the lesion part are extracted by the extraction unit 24A using image recognition processing by an artificial intelligence (AI) system and/or a pattern matching system is exemplified.

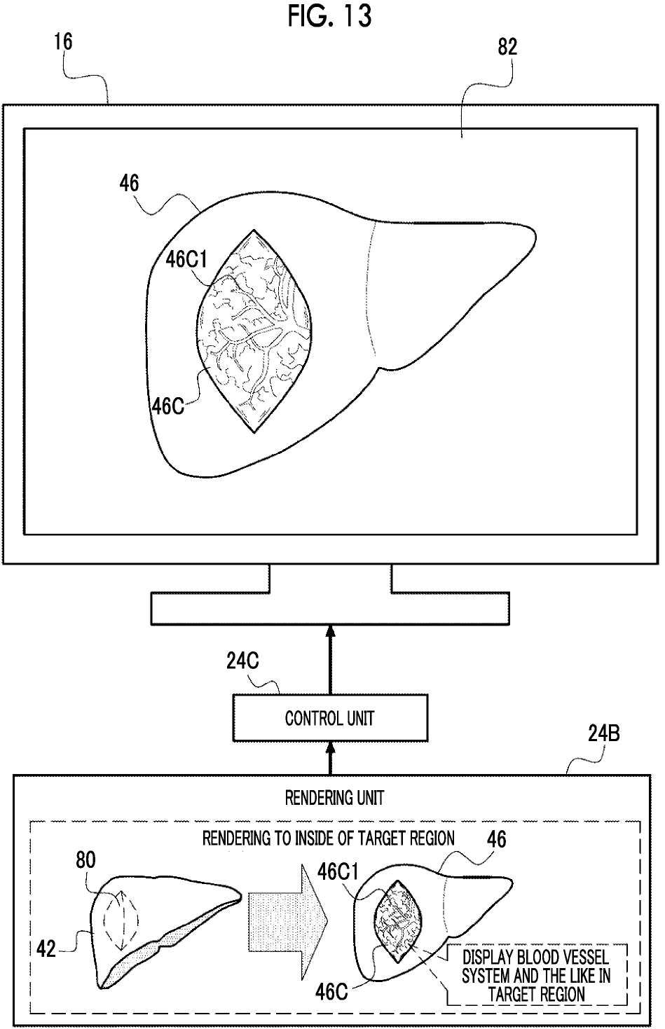

The rendering unit 24B generates a rendering image 46 in a state in which the target region 80 is brought into non-display, and the blood vessel system, the lymphatic system, the nervous system, and/or the lesion part included in the target region 80 are displayed. In this case, the target region 80 is a region where at least one of the region showing the blood vessel system, the region showing the lymphatic system, the region showing the nervous system, or the region showing the lesion part is excluded. As shown in FIG. 13 as an example, the rendering unit 24B performs rendering to the inside of the target region 80 based on the regions specified by the region specification unit 24G showing the blood vessel system, the lymphatic system, the nervous system, and/or the lesion part, respectively. With this, a rendering image 46C1 is generated in a region 46C in the rendering image 46 corresponding to the inside of the target region 80. As the rendering image 46C1, a rendering image 46C1 in a state in which the target region 80 is brought into non-display, and the regions showing the blood vessel system, the lymphatic system, the nervous system, and/or the lesion part, respectively, are displayed.

The control unit 24C performs display control such that the rendering image 46 including the rendering image 46C1 is displayed on the display 16. As shown in FIG. 13 as an example, on the screen 82, the rendering image 46 including the rendering image 46C1 is displayed.

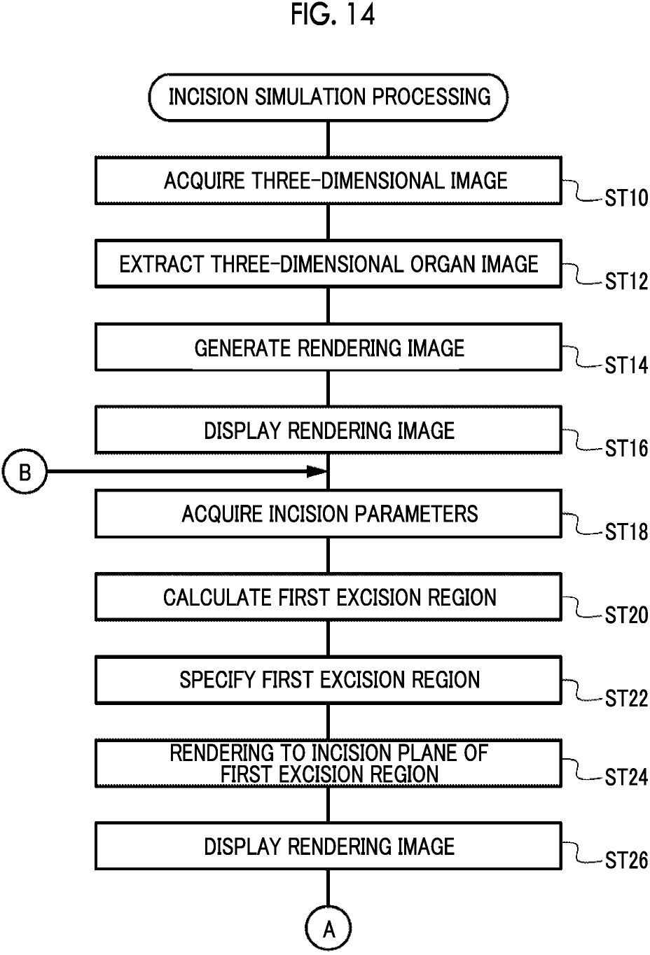

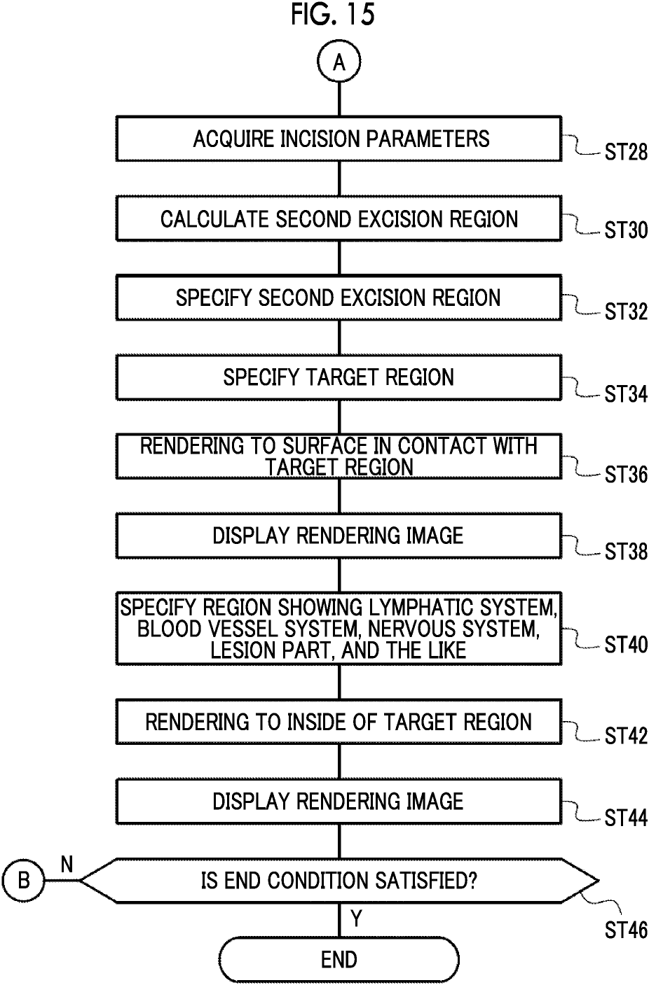

Next, the operations of the medical service support device 10 will be described with reference to FIGS. 14 and 15.

FIGS. 14 and 15 show an example of a flow of incision simulation processing that is executed by the processor 24. The flow of the incision simulation processing shown in FIGS. 14 and 15 is an example of an “incision simulation method” according to the technique of the present disclosure.

In the incision simulation processing shown in FIG. 14, first, in Step ST10, the extraction unit 24A acquires the three-dimensional image 38 from the storage 26 (see FIG. 3). After the processing of Step ST10 is executed, the incision simulation processing proceeds to Step ST12.

In Step ST12, the extraction unit 24A extracts the three-dimensional organ image 42 from the three-dimensional image 38 acquired in Step ST10 (see FIG. 3). After the processing of Step ST12 is executed, the incision simulation processing proceeds to Step ST14.

In Step ST14, the rendering unit 24B performs rendering to the three-dimensional organ image 42 extracted in Step ST12 to generate the rendering image 46 (see FIGS. 4 and 5). After the processing of Step ST14 is executed, the incision simulation processing proceeds to Step ST16.

In Step ST16, the control unit 24C displays the rendering image 46 generated in Step ST14 on the display 16 (see FIGS. 6 and 7). After the processing of Step ST16 is executed, the incision simulation processing proceeds to Step ST18.

In Step ST18, the incision parameter acquisition unit 24D acquires the incision parameters (that is, the first incision line 60 and the first depth 64) received by the reception device 14 (see FIG. 8). After the processing of Step ST18 is executed, the incision simulation processing proceeds to Step ST20.

In Step ST20, the first excision region calculation unit 24E calculates the first excision region 65 based on the incision parameters acquired in Step ST18 (see FIG. 8). After the processing of Step ST20 is executed, the incision simulation processing proceeds to Step ST22.

In Step ST22, the region specification unit 24G specifies the first excision region 65 from the three-dimensional organ image 42 extracted in Step ST12 using the first excision region 65 calculated in Step ST20 (see FIG. 8). After the processing of Step ST22 is executed the incision simulation processing proceeds to Step ST24.

In Step ST24, the rendering unit 24B performs rendering to the first excision region 65 specified in Step ST22 to generate the rendering image 46B1. After the processing of Step ST24 is executed, the incision simulation processing proceeds to Step ST26.

In Step ST26, the control unit 24C displays the rendering image 46 including the rendering image 46B1 generated in Step ST24 on the display 16. After the processing of Step ST26 is executed, the incision simulation processing proceeds to Step ST28 in the flow of the incision simulation processing shown in FIG. 15 as an example.

In the incision simulation processing shown in FIG. 15, in Step ST28, the incision parameter acquisition unit 24D acquires the incision parameters (that is, the second incision line 70 and the second depth 74) received by the reception device 14 (see FIG. 11). After the processing of Step ST28 is executed, the incision simulation processing proceeds to Step ST30.

In Step ST30, the second excision region calculation unit 24F calculates the second excision region 75 based on the incision parameters acquired in Step ST28 (see FIG. 11). After the processing of Step ST30 is executed, the incision simulation processing proceeds to Step ST32.

In Step ST32, the region specification unit 24G specifies the second excision region 75 from the three-dimensional organ image 42 extracted in Step ST12 using the second excision region 75 calculated in Step ST32 (see FIG. 11). After the processing of Step ST32 is executed, the incision simulation processing proceeds to Step ST34.

In Step ST34, the region specification unit 24G specifies the target region 80 included in the first excision region 65 specified in the Step ST22 and the second excision region 75 specified in Step ST32. After the processing of Step ST34 is executed, the incision simulation processing proceeds to Step ST36.

In Step ST36, the rendering unit 24B performs rendering to the three-dimensional organ image 42 in a state in which the target region 80 is excluded, based on the target region 80 specified in Step ST34 to generate the rendering image 46B2. After the processing of Step ST36 is executed, the incision simulation processing proceeds to Step ST38.

In Step ST38, the control unit 24C displays the rendering image 46 including the rendering image 46B2 generated in Step ST36 on the display 16. After the processing of Step ST38 is executed, the incision simulation processing proceeds to Step ST40.

In Step ST40, the region specification unit 24G specifies the regions including the blood vessel system, the lymphatic system, the nervous system, and/or the lesion part included in the target region 80 specified in Step ST34. After the processing of Step ST40 is executed, the incision simulation processing proceeds to Step ST42.

In Step ST42, the rendering unit 24B performs rendering to the regions specified in Step ST40 including the blood vessel system, the lymphatic system, the nervous system, and/or the lesion part to generate the rendering image 46C1. After the processing of Step ST42 is executed, the incision simulation processing proceeds to Step ST44.

In Step ST44, the control unit 24C displays the rendering image 46 including the rendering image 46C1 generated in Step ST42 on the display 16. After the processing of Step ST42 is executed, the incision simulation processing proceeds to Step ST46.

In Step ST46, the control unit 24C determines whether or not a condition (hereinafter, referred to as an “end condition”) for ending the incision simulation processing is satisfied. Examples of the end condition include a condition that an instruction to end the incision simulation processing is received by the reception device 14. In Step ST46, in a case where the end condition is not satisfied, determination is negative, and the incision simulation processing proceeds to Step ST18. In Step ST46, in a case where the end condition is satisfied, determination is affirmative, and the incision simulation processing ends.

As described above, in the medical service support device 10, the first excision region 65 is calculated, and the second excision region 75 is calculated based on the first excision region 65. Therefore, according to this configuration, it is possible to specify an inside of an excision region obtained by continuous incision.

In the medical service support device 10, the second excision region 75 is calculated based on the third depth 77 that is the depth based on the depth of the first excision region 65 and the second depth 74 at the designated position on the second incision line 70. Therefore, according to this configuration, the second excision region 75 can be simply and easily calculated compared to a case where the second excision region 75 is calculated without using the third depth 77. That is, the second excision region 75 is calculated based on the depth of the first excision region 65 and the second depth 74 at the designated position, regardless of the shape of the first excision region 65. Therefore, a calculation cost for calculating the second excision region 75 is reduced compared to a case where the second excision region 75 is calculated taking into consideration a space after the first excision region 65 is excised.

In the medical service support device 10, since the third depth 77 is the sum of the depth of the first excision region 65 and the second depth 74, the second excision region 75 is simply and easily calculated compared to a case where the third depth 77 is defined as a value other than the sum of the second depth 74 and the depth of the first excision region 65.

In the medical service support device 10, the depth at each position of the second incision line 70 is calculated based on the third depth 77, and the second excision region 75 is calculated based on the depth at each position of the second incision line 70. Therefore, according to this configuration, the second excision region 75 can be simply and easily calculated compared to a case where the second excision region 75 is calculated without using the third depth 77.

In the medical service support device 10, the depth at each position of the second incision line 70 is shallower from the designated position toward the end of the second incision line 70 at each position on the second incision line 70. Therefore, according to this configuration, the second excision region 75 is calculated with high accuracy compared to a case where the depth at each position of the second incision line 70 is constant.

In the medical service support device 10, the second incision width 79 at each position of the second incision line 70 is calculated based on the depth at each position of the second incision line 70, and the second excision region 75 is calculated based on the second incision width 79. Therefore, according to this configuration, the second excision region 75 is calculated compared to a case where the second excision region 75 is calculated without using the second incision width 79.

In the medical service support device 10, the second incision width 79 at each position of the second incision line 70 is narrower from the designated position toward the end of the second incision line 70 at each position on the second incision line 70. Therefore, according to this configuration, the second excision region 75 is calculated with high accuracy compared to a case where the second incision width 79 at each position of the second incision line 70 is constant.

In the medical service support device 10, the depth at each position of the second incision width is shallower from the position on the second incision line 70 toward the end of the second incision width 79, and the second excision region 75 is calculated based on the depth at each position of the second incision width 79. Therefore, according to this configuration, the second excision region 75 is calculated with high accuracy compared to a case where the depth at each position of the second incision width 79 is constant.

In the medical service support device 10, the designated position on the second incision line 70 is the intermediate portion of the second incision line 70. Therefore, according to this configuration, the second excision region 75 is simply and easily calculated compared to a case where the third depth 77 is a depth defined based on the depth of the first excision region 65 and the second depth 74 at a position other than the intermediate portion of the second incision line 70.

In the medical service support device 10, the designated position on the second incision line 70 is the middle point of the second incision line 70. Therefore, according to this configuration, the second excision region 75 is simply and easily calculated compared to a case where the third depth 77 is a depth defined based on the depth of the first excision region 65 and the second depth 74 at a position other than the middle point of the second incision line 70.

In the medical service support device 10, the depth at each position of the first incision line 60 is calculated based on the first depth 64, and the first excision region 65 is calculated based on the depth at each position of the first incision line 60. Therefore, according to this configuration, the first excision region 65 is simply and easily calculated compared to a case where first excision region 65 is calculated without using the depth at each position of the first incision line 60. Since the way of calculation of the depth at each position of the first incision line 60 is common to the case of the second incision line 70, a calculation cost is reduced compared to a case where different calculation methods are used between the first incision line 60 and the second incision line 70.

In the medical service support device 10, the depth at each position of the first incision line 60 is shallower from the position of the first depth 64 toward the end of the first incision line 60 at each position on the first incision line 60. Therefore, according to this configuration, the first excision region 65 is calculated with high accuracy compared to a case where the depth at each position of the first incision line 60 is constant. Since a way of change of the depth at each position of the first incision line 60 is common to a case of the second incision line 70, a calculation cost is reduced compared to a case where a way of change is different between the first incision line 60 and the second incision line 70.

In the medical service support device 10, the first incision width 67 at each position of the first incision line 60 is calculated based on the depth at each position of the first incision line 60, and the first excision region 65 is calculated based on the first incision width 67. Therefore, according to this configuration, the first excision region 65 is simply and easily calculated compared to a case where the first excision region 65 is calculated without using the first incision width 67. Since the way of calculation of the first incision width 67 is common to a case of the second incision width 79, a calculation cost is reduced compared to the case where different calculation methods are used between the first incision width 67 and the second incision width 79.

In the medical service support device 10, the first incision width 67 at each position of the first incision line 60 is narrower from the position of the first depth 64 toward the end of the first incision line 60 at each position on the first incision line 60. Therefore, according to this configuration, the first excision region 65 is calculated with high accuracy compared to a case where the first incision width 67 at each position of the first incision line 60 is constant. Since a way of change of the first incision width 67 is common to a case of the second incision width 79, a calculation cost is reduced compared to a case where a way of change is different between the first incision width 67 and the second incision width 79.

In the medical service support device 10, the depth at each position of the first incision width 67 is shallower from the position on the first incision line 60 toward the end of the first incision width 67, and the first excision region 65 is calculated based on the depth at each position of the first incision width 67. Therefore, according to this configuration, the first excision region 65 is calculated compared to a case where the depth at each position of the first incision width 67 is constant. Since a way of change of the depth at each position of the first incision width 67 is common to a case of the second incision width 79, a calculation cost is reduced compared to a case where a way of change is different between the first incision width 67 and the second incision width 79.

In the medical service support device 10, the position of the first depth 64 of the first incision line 60 is the intermediate portion of the first incision line 60. Therefore, according to this configuration, the first excision region 65 is simply and easily calculated compared to a case where the first depth 64 is a depth defined at a position other than the intermediate portion of the first incision line 60.

In the medical service support device 10, the position of the first depth 64 of the first incision line 60 is the middle point of the first incision line 60. Therefore, according to this configuration, the first excision region 65 is simply and easily calculated compared to a case where the first depth 64 is a depth defined at a position other than the middle point of the first incision line 60.

In the medical service support device 10, the target region 80 is the region where at least one of the region showing the blood vessel system, the region showing the lymphatic system, the region showing the nervous system, or the region showing the lesion part is excluded. Therefore, according to this configuration, it is possible to make the user 18 visually recognize at least one of the region showing the blood vessel system, the region showing the lymphatic system, the region showing the nervous system, or the region showing the lesion part, in the region after continuous incision.

In the above-described embodiment, although a form example where the continuous incision simulation processing is executed in which the first excision region 65 is calculated based on the first incision line 60 and the first depth 64, and the second excision region 75 is calculated based on the second incision line 70 and the third depth 77 has been described, the technique of the present disclosure is not limited thereto. For example, continuous incision simulation processing in a case where three times or more of incision are performed may be executed.

In the above-described embodiment, although a form example where the first excision region 65 is included in the second excision region 75 as shown in FIG. 11 has been described, the first excision region 65 and the second excision region 75 may have an overlapping portion as viewed from a depth direction, and the technique of the present disclosure is not limited thereto. For example, as the first excision region 65 and the second excision region 75 are viewed from the depth direction, the regions of the first excision region 65 and the second excision region 75 may deviate from each other.

In the above-described embodiment, although a form example where the depth at each position of the first incision line 60 and the second incision line 70 (hereinafter, simply referred to as “incision line”), change in first incision width 67 and second incision width 79 (hereinafter, simply referred to as “incision width”), the depth at each position of the incision width, or the first excision region 65 and the second excision region 75 (hereinafter, simply referred to as “excision region”) are obtained using the arithmetic expressions based on the incision parameters received through the reception device 14 has been described, the technique of the present disclosure is not limited thereto. For example, various tables that have the incision parameters as input values and have the depth at each position of the incision line, the change in incision width, the depth at each position of the incision width, or the excision region as an output value may be used.

In the above-described embodiment, although a form example where the depth at each position of the incision line, the incision width, and the depth at each position of the incision width is changed monotonously has been described, the technique of the present disclosure is not limited thereto. For example, the depth at each position of the incision line, the incision width, and/or the depth each position of the incision width may be a constant value in a certain region and is changed monotonously from an end portion of the region where the value is constant.

In the above-described embodiment, although a form example where the incision parameters are received through the reception device 14 has been described, the technique of the present disclosure is not limited thereto. For example, the incision parameters may be determined depending on various conditions (for example, types of organs) or may be values that satisfy conditions given to an incision target object. Various arithmetic expressions that calculate the excision region based on the depth at each position of the incision line, the incision width, and the depth at each position of the incision width in the above-described embodiment have different arithmetic expressions depending on various conditions, and arithmetic expressions may be selected based on various conditions. For example, different arithmetic expressions may be provided for types of organs, and an arithmetic expression corresponding to a target organ of the three-dimensional organ image 42 may be selected. In the above-described embodiment, although a form example where the input of the incision line and the depth of incision as the incision parameters is received through the reception device 14 has been described, the technique of the present disclosure is not limited thereto. For example, a plurality of relational expressions of the incision line and the depth of incision are provided depending on various conditions (for example, types of organs), only an input of any one of the incision line or the depth of incision may be received, and the other value may be calculated using a relational expression of the incision line and the depth of incision depending on a condition that the three-dimensional image 38 or the three-dimensional organ image 42 satisfies. For example, a plurality of relational expressions of the incision line and the depth of incision for organs may be held, only an input of the incision line may be received through the reception device 14, and the depth of incision may be calculated using a relational expression of the incision line and the depth of incision corresponding to a target organ of the three-dimensional organ image 42.

In the above-described embodiment, although a form example where the first excision region 65 is calculated based on the depth at each position of the first incision line 60, the first incision width 67, and the depth at each position of the first incision width 67 in addition to first incision line 60 and the first depth 64 has been described, the technique of the present disclosure is not limited thereto. For example, in a case of calculating the first excision region 65, any one of the depth at each position of the first incision line 60, the first incision width 67, or the depth at each position of the first incision width 67 may be used or two parameters may be used in combination. The first excision region 65 may be calculated only from the first incision line 60 and the first depth 64.

In the above-described embodiment, although a form example where the second excision region 75 is calculated based on the depth at each position of the second incision line 70, the second incision width 79, and the depth at each position of the second incision width 79 in addition to the second incision line 70 and the third depth 77 has been described, the technique of the present disclosure is not limited thereto. For example, in a case of calculating the second excision region 75, any one of the depth at each position of the second incision line 70, the second incision width 79, or the depth at each position of the second incision width 79 may be used or two parameters may be used in combination. The second excision region 75 may be calculated only from the second incision line 70 and the third depth 77.

In the above-described embodiment, although a form example where the target region 80 is the same as the second excision region 75 has been described, the technique of the present disclosure is not limited thereto. The target region 80 may be included in the first excision region 65 and the second excision region 75 or the target region 80 may be a part of the first excision region 65 and/or the second excision region 75.

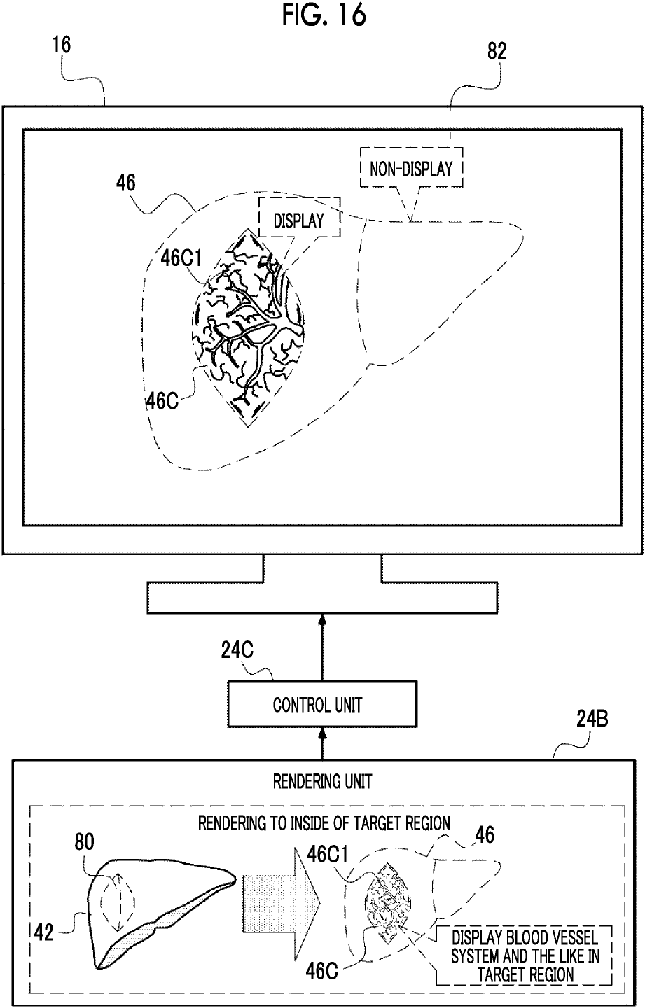

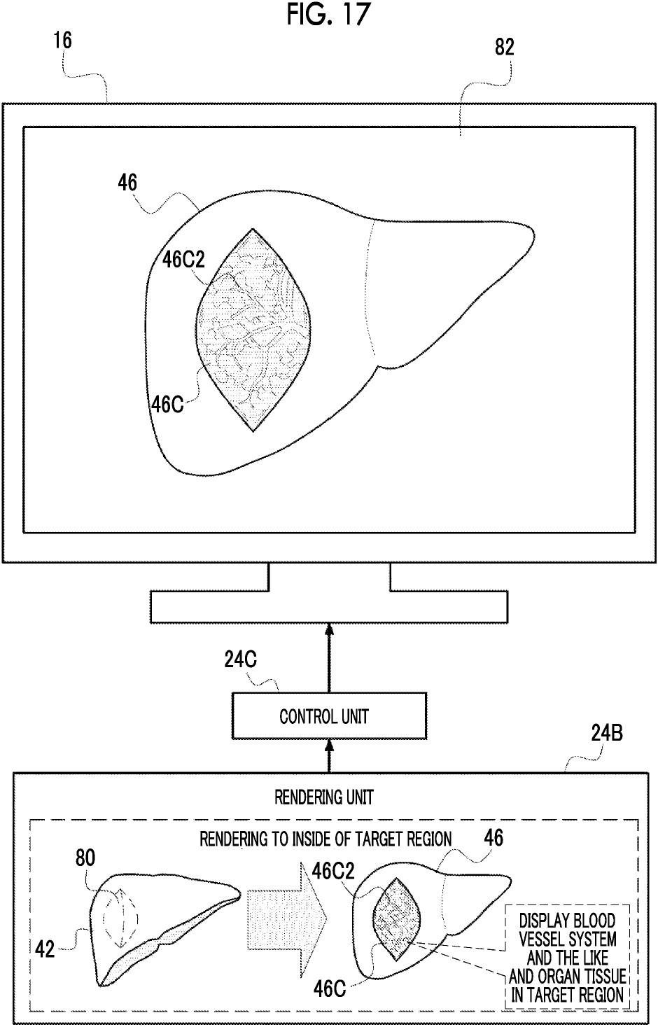

In the above-described embodiment, although a form example where the regions showing the blood vessel system, the lymphatic system, the nervous system, and/or the lesion part inside of the target region 80 are displayed in the region 46C of the rendering image 46 has been described, the technique of the present disclosure is not limited thereto. As shown in FIG. 16 as an example, only the rendering image 46C1 showing the blood vessel system, the lymphatic system, the nervous system, and/or the lesion part may be displayed on the screen 82. As shown in FIG. 17 as an example, a rendering image 46C2 showing an organ tissue (that is, a tissue included in the target region 80 other than the blood vessel system and the like) in addition to the blood vessel system, the lymphatic system, the nervous system, and/or the lesion part may be displayed on the screen 82.

In the above-described embodiment, although a form example where the rendering image 46 shown in FIG. 12 as an example in which the target region 80 is brought into non-display, and the rendering image 46 shown in FIG. 13 as an example in which the regions showing the blood vessel system, the lymphatic system, the nervous system, and/or the lesion part are displayed are shown has been described, the technique of the present disclosure is not limited thereto. For example, only one of the rendering image 46 shown in FIG. 12 in which the target region 80 is brought into non-display or the rendering image 46 shown in FIG. 13 in which the regions showing the blood vessel system, the lymphatic system, the nervous system, and/or the lesion part are displayed may be displayed, or the two rendering images 46 may be switched. For example, an aspect may be made in which only the rendering image 46 in which the target region 80 is brought into non-display is shown without executing processing of showing the rendering image 46 in which the regions showing the blood vessel system, the lymphatic system, the nervous system, and/or the lesion part are displayed.

For example, the rendering image 46 shown in FIGS. 12, 13, 16, or 17 may be switched and displayed. The control unit 24C performs display control based on an instruction received through the reception device 14 such that the display 16 switches the screen 82 showing the rendering image 46 shown in FIGS. 12, 13, 16, or 17.

In the above-described embodiment, although a form example where the incision simulation processing is executed by the processor 24 of the image processing device 12 included in the medical service support device 10 has been described, the technique of the present disclosure is not limited thereto, and a device that executes the incision simulation processing may be provided outside the medical service support device 10.

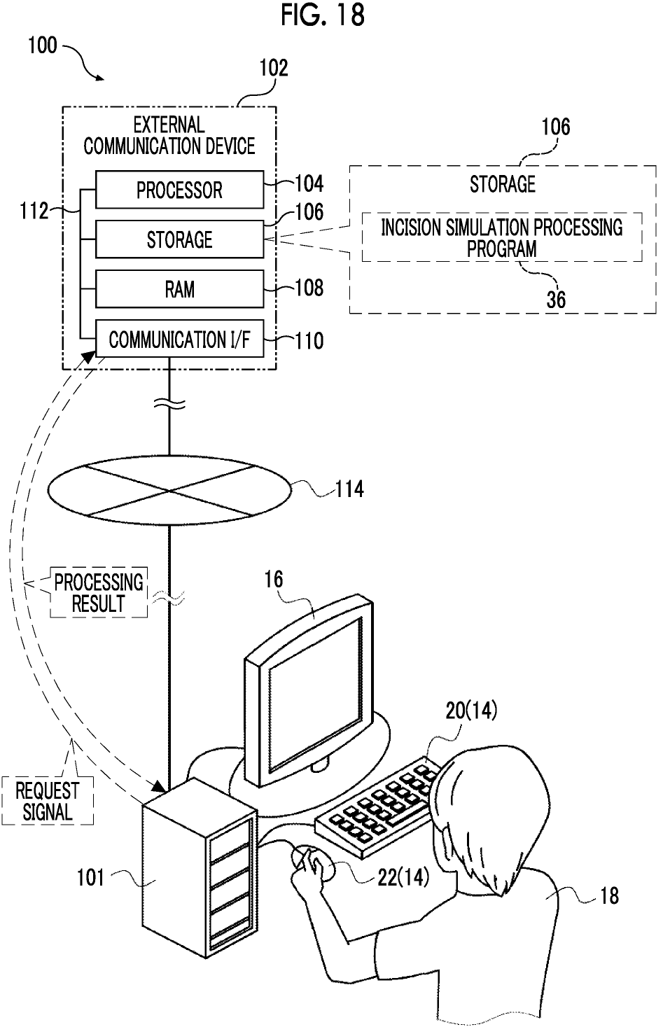

In this case, as shown in FIG. 18 as an example, a medical service support system 100 may be used. The medical service support system 100 comprises an information processing device 101 and an external communication device 102. The information processing device 101 is a device in which the incision simulation processing program 36 is removed from the storage 26 of the image processing device 12 that is included in the medical service support device 10 described in the above-described embodiment. The external communication device 102 is, for example, a server. The server is realized by, for example, a main frame. Here, although the main frame has been illustrated, this is merely an example, and the server may be realized by cloud computing or may be realized by network computing, such as fog computing, edge computing, or grid computing. Here, although the server is illustrated as an example of the external communication device 102, this is merely an example, and instead of the server, at least one personal computer or the like may be used as the external communication device 102.

The external communication device 102 comprises a processor 104, a storage 106, a RAM 108, and a communication I/F 110, and the processor 104, the storage 106, the RAM 108, and the communication I/F 110 are connected by a bus 112. The communication I/F 110 is connected to the information processing device 101 through a network 114. The network 114 is, for example, the Internet. The network 114 is not limited to the Internet, and may be a WAN and/or a LAN, such as an intranet.

The incision simulation processing program 36 is stored in the storage 106. The processor 104 executes the incision simulation processing program 36 on the RAM 108. The processor 104 executes the above-described incision simulation processing following the incision simulation processing program 36 that is executed on the RAM 108.

The information processing device 101 transmits a request signal for requesting the execution of the incision simulation processing to the external communication device 102. The communication I/F 110 of the external communication device 102 receives the request signal through the network 114. The processor 104 executes the incision simulation processing following the incision simulation processing program 36 and transmits a processing result to the information processing device 101 through the communication I/F 110. The information processing device 101 receives the processing result (for example, a processing result by the region specification unit 24G) transmitted from the external communication device 102 with the communication I/F 30 (see FIG. 2) and outputs the received processing result to various devices, such as the display 16.

In the example shown in FIG. 18, the external communication device 102 is an example of an “incision simulation device” according to the technique of the present disclosure, and the processor 104 is an example of a “processor” according to the technique of the present disclosure.

The incision simulation processing may be distributed to and executed by a plurality of devices including the information processing device 101 and the external communication device 102. In the above-described embodiment, although the three-dimensional image 38 is stored in the storage 26 of the medical service support device 10, an aspect may be made in which the three-dimensional image 38 is stored in the storage 106 of the external communication device 102 and is acquired from the external communication device 102 through the network at a timing at which the incision simulation processing is executed.

In the above-described embodiment, although a form example where the processor 24 is realized by a CPU and a GPU has been described, the technique of the present disclosure is not limited thereto, and the processor 24 may be a processor that is realized by at least one CPU, at least one GPU, at least one general-purpose computing on graphics processing units (GPGPU), and/or at least one Tensor processing unit (TPU).

In the above-described embodiment, although a form example where the incision simulation processing program 36 is stored in the storage 26 has been described, the technique of the present disclosure is not limited thereto. For example, the incision simulation processing program 36 may be stored in a storage medium (not shown), such as an SSD or a USB memory. The storage medium is a portable non-transitory storage medium. The incision simulation processing program 36 stored in the storage medium is installed on the image processing device 12 of the medical service support device 10. The processor 24 executes the incision simulation processing following the incision simulation processing program 36.

The incision simulation processing program 36 may be stored in a storage device of another computer, a server device, or the like connected to the medical service support device 10 through the network (not shown), and the incision simulation processing program 36 may be downloaded in response to a request of the medical service support device 10 and may be installed on the image processing device 12. That is, the program (program product) described in the present embodiment may be provided by a recording medium or may be distributed from an external computer.

The entire incision simulation processing program 36 may not be stored in the storage device of another computer, the server device, or the like connected to the medical service support device 10 or the storage 26, and a part of the incision simulation processing program 36 may be stored. The storage medium, the storage device of another computer, the server device, or the like connected to the medical service support device 10, and other external storages (for example, a database) may be placed as a memory that is connected to the processor 24 directly or indirectly and used.

In the above-described embodiment, although the image processing device 12 is illustrated as a computer, the technique of the present disclosure is not limited thereto, and instead of the computer, a device including an application specific integrated circuit (ASIC), a field-programmable gate array (FPGA), and/or a programmable logic device (PLD) may be applied. Instead of the computer, a combination of a hardware configuration and a software configuration may be used.

As a hardware resource for executing the incision simulation processing described in the above-described embodiment, various processors described below can be used. Examples of the processors include a CPU that is a general-purpose processor configured to execute software, that is, a program to function as the hardware resource for executing the incision simulation processing. Examples of the processors include a dedicated electric circuit that is a processor, such as an FPGA, a PLD, or an ASIC, having a circuit configuration dedicatedly designed for executing specific processing. A memory is embedded in or connected to any processor, and any processor uses the memory to execute the incision simulation processing.

The hardware resource for executing the incision simulation processing may be configured with one of various processors or may be configured with a combination of two or more processors (for example, a combination of a plurality of FPGAs or a combination of a CPU and an FPGA) of the same type or different types. The hardware resource for executing the incision simulation processing may be one processor.

As an example where the hardware resource is configured with one processor, first, there is a form in which one processor is configured with a combination of one or more CPUs and software, and the processor functions as the hardware resource for executing the incision simulation processing. Second, as represented by System-on-a-chip (SoC) or the like, there is a form in which a processor that realizes all functions of a system including a plurality of hardware resources for executing the incision simulation processing into one integrated circuit (IC) chip is used. In this way, the incision simulation processing is realized using one or more processors among various processors described above as a hardware resource.

As the hardware structures of various processors, more specifically, an electric circuit in which circuit elements, such as semiconductor elements, are combined can be used. The above-described incision simulation processing is merely an example. Accordingly, it goes without saying that unnecessary steps may be deleted, new steps may be added, or a processing order may be changed without departing from the gist.

The content of the above description and the content of the drawings are detailed description of portions according to the technique of the present disclosure, and are merely examples of the technique of the present disclosure. For example, the above description relating to configuration, function, operation, and advantageous effects is description relating to configuration, function, operation, and advantageous effects of the portions according to the technique of the present disclosure. Thus, it goes without saying that unnecessary portions may be deleted, new elements may be added, or replacement may be made to the content of the above description and the content of the drawings without departing from the gist of the technique of the present disclosure. Furthermore, to avoid confusion and to facilitate understanding of the portions according to the technique of the present disclosure, description relating to common technical knowledge and the like that does not require particular description to enable implementation of the technique of the present disclosure is omitted from the content of the above description and the content of the drawings.

In the specification, “A and/or B” is synonymous with “at least one of A or B”. That is, “A and/or B” may refer to A alone, B alone, or a combination of A and B. Furthermore, in the specification, a similar concept to “A and/or B” applies to a case in which three or more matters are expressed by linking the matters with “and/or”.

All cited documents, patent applications, and technical standards described in the specification are incorporated by reference in the specification to the same extent as in a case where each individual cited document, patent application, or technical standard is specifically and individually indicated to be incorporated by reference.

What is claimed is:

1. An incision simulation device comprising:
a processor that is configured to:
acquire a first incision line for a three-dimensional organ image that is a three-dimensional image showing an organ,
acquire a first depth of incision to the first incision line,
calculate a first excision region based on the first incision line and the first depth,
acquire a second incision line for the three-dimensional organ image,
acquire a second depth of incision to the second incision line,
calculate a second excision region based on the first excision region, the second incision line, and the second depth, and
identify a first region included in the first excision region and the second excision region, in the three-dimensional organ image.

2. The incision simulation device according to claim 1,
wherein the processor is configured to calculate the second excision region based on a third depth and the second incision line, and
the third depth is a depth based on a depth of the first excision region and the second depth at a designated position on the second incision line.

3. The incision simulation device according to claim 2,
wherein the third depth is a sum of the depth of the first excision region and the second depth.

4. The incision simulation device according to claim 2,
wherein the processor is configured to
calculate a depth at each position of the second incision line based on the third depth, and
calculate the second excision region based on the depth at each position of the second incision line.

5. The incision simulation device according to claim 4,
wherein the depth at each position of the second incision line is shallower from the designated position toward an end of the second incision line at each position on the second incision line.

6. The incision simulation device according to claim 4,
wherein the processor is configured to
calculate a second incision width at each position of the second incision line based on the depth at each position of the second incision line, and
calculate the second excision region based on the second incision width.

7. The incision simulation device according to claim 6,
wherein the second incision width at each position of the second incision line is narrower from the designated position toward an end of the second incision line at each position on the second incision line.

8. The incision simulation device according to claim 6,
wherein a depth at each position of the second incision width is shallower from a position on the second incision line toward an end of the second incision width, and
the processor is configured to calculate the second excision region based on the depth at each position of the second incision width.

9. The incision simulation device according to claims 2,
wherein the designated position on the second incision line is an intermediate portion of the second incision line.

10. The incision simulation device according to claim 9,
wherein the designated position on the second incision line is a middle point of the second incision line.

11. The incision simulation device according to claims 1,
wherein the processor is configured to
calculate a depth at each position of the first incision line based on the first depth, and
calculate the first excision region based on the depth at each position of the first incision line.

12. The incision simulation device according to claim 11,
wherein the depth at each position of the first incision line is shallower from a position of the first depth toward an end of the first incision line at each position on the first incision line.

13. The incision simulation device according to claim 11,
wherein the processor is configured to
calculate a first incision width at each position of the first incision line based on the depth at each position of the first incision line, and
calculate the first excision region based on the first incision width.

14. The incision simulation device according to claim 13,
wherein the first incision width at each position of the first incision line is narrower from a position of the first depth toward an end of the first incision line at each position on the first incision line.

15. The incision simulation device according to claim 13,
wherein a depth at each position of the first incision width is shallower from a position on the first incision line toward an end of the first incision width, and
the processor is configured to calculate the first excision region based on the depth at each position of the first incision width.

16. The incision simulation device according to claims 12,
wherein the position of the first depth of the first incision line is an intermediate portion of the first incision line.

17. The incision simulation device according to claim 16,
wherein the position of the first depth of the first incision line is a middle point of the first incision line.

18. The incision simulation device according to claims 1,
wherein the first region is a region where at least one of a region showing a blood vessel system, a region showing a lymphatic system, a region showing a nervous system, or a region showing a lesion part is excluded.

19. An incision simulation method comprising:
acquiring a first incision line for a three-dimensional organ image that is a three-dimensional image showing an organ;
acquiring a first depth of incision to the first incision line;
calculating a first excision region based on the first incision line and the first depth;
acquiring a second incision line for the three-dimensional organ image;
acquiring a second depth of incision to the second incision line;
calculating a second excision region based on the first excision region, the second incision line, and the second depth; and
identifying a first region included in the first excision region and the second excision region, in the three-dimensional organ image.

20. A non-transitory storage medium storing a program that causes a computer to execute a process, the process comprising:
acquiring a first incision line for a three-dimensional organ image that is a three-dimensional image showing an organ;
acquiring a first depth of incision to the first incision line;
calculating a first excision region based on the first incision line and the first depth;
acquiring a second incision line for the three-dimensional organ image;
acquiring a second depth of incision to the second incision line;
calculating a second excision region based on the first excision region, the second incision line, and the second depth; and
identifying a first region included in the first excision region and the second excision region, in the three-dimensional organ image.
